(19) United States
(12) Patent Application Publication
(10) Pub. No.: US 2014/0044736 A1
(43) Pub. Date: Feb. 13, 2014

(54) PROCOLLAGEN CARBOXY-TERMINAL PROPEPTIDES AS A TARGET AND TREATMENT FOR ANGIOGENESIS RELATED DISEASES

(71) Applicant: Hans-Joerg Gerg Hammers, Laurel, MD (US)

(72) Inventor: Hans-Joerg Gerg Hammers, Laurel, MD (US)

(73) Assignee: The Johns Hopkins University, Baltimore, MD (US)

(21) Appl. No.: 14/003,938

(22) PCT Filed: Mar. 8, 2012
(86) PCT No.: PCT/US2012/028193

§ 371 Date: Oct. 21, 2013

Related U.S. Application Data
(60) Provisional application No. 61/450,445, filed on Mar. 8, 2011.

(51) Int. Cl. A61K 39/395; A61K 38/18; A61K 31/713; A61K 38/39

(52) U.S. Cl. CPC A61K 39/3955; A61K 38/39; A61K 38/1866; A61K 31/713
USPC 424/172.1; 514/17.2; 514/8.1; 514/44 R; 514/44 A; 435/375; 536/24.5; 536/23.1; 435/320.1; 435/366

(57) ABSTRACT

The present invention relates to the field of angiogenesis. More specifically, the present invention provides methods and compositions for modulating angiogenesis. In a specific embodiment, a method for modulating a blood vessel in a subject in need thereof comprising contacting a cell of the subject with a procollagen carboxy-terminal propeptide, a biologically active fragment or mimetic thereof, thereby modulating the blood vessel.

### CROSS-REFERENCE TO RELATED APPLICATIONS

This application claims the benefit of U.S. Provisional Application No. 61/450,445, filed Mar. 8, 2011; which is incorporated herein by reference in its entirety.

### FIELD OF THE INVENTION

The present invention relates to the field of angiogenesis. More specifically, the present invention provides methods and compositions for modulating angiogenesis.

### INCORPORATION-BY-REFERENCE OF MATERIAL SUBMITTED ELECTRONICALLY

This application contains a sequence listing. It has been submitted electronically via EFS-Web as an ASCII text file entitled “P11455-02_ST25.txt.” The sequence listing is 115,621 bytes in size, and was created on Mar. 7, 2012. It is hereby incorporated by reference in its entirety.

### BACKGROUND OF THE INVENTION

Angiogenesis is an established treatment modality for solid tumors and several antiangiogenic agents have been approved for clinical use by the regulatory authorities. Virtually all of these drugs are targeting the vascular endothelial growth factor (VEGF) pathway and display varying degrees of clinical activity. One of the most sensitive tumor types is clear-cell renal cell carcinoma (ccRCC), which also has some of the highest VEGF expression levels. In fact, ccRCC is typically sensitive enough that it can be treated with single agents (e.g. the tyrosine kinase inhibitor sunitinib) and often leads to tumor responses. This is quite impressive considering that kidney cancer is notoriously resistant to traditional cytotoxic chemotherapy. Importantly, it illustrates the potential of effective antiangiogenic therapy, which can be observed when the strategy (VEGF inhibition) matches the molecular underpinning of the cancer. In the case of ccRCC it is the unique overexpression and addiction to the VEGF pathway, which renders this tumor type so susceptible to VEGF pathway. Therefore, it is not surprising that the response in other tumor types is less impressive and the combination with cytotoxic chemotherapy is required. After almost a decade of anti-VEGF and “me too”-anti-VEGF therapy, the field of tumor angiogenesis inhibition is at a crossroads and there is a need to develop more effective antiangiogenic therapies.

### SUMMARY OF THE INVENTION

The present invention is based, at least in part, on the identification of a stromal derived factor, the c-terminal propeptide (PICP: procollagen I carboxyterminal peptide) of the collagen I alpha1 (COLA1A1) gene, which facilitates lumenized sprouting in the presence of proangiogenic growth factors. The human form is comprised of amino acids 1219 through 1464, and mouse fibroblasts and other mouse cells produce the same activity. Indeed, a large degree of homology among different species is anticipated. More importantly, this factor will be produced by any collagen I producing cell (e.g., fibroblasts, myofibroblasts, osteoblasts) suggesting that any active, healing, stimulated or cancerous tissues can produce this molecule and, therefore, facilitate efficient angiogenesis. Given the expected expression pattern in growing or activated tissues, the present inventors believe that this molecule is a fundamental component of the so-called angiogenic switch.

As described herein, PICP facilitates the formation of lumenized vessel-like structures in three-dimensional extracellular matrices. Derived from stromal cells such as fibroblasts, this discovery has profound implications on either targeting pathological angiogenesis such as cancer and age-related macular degeneration or to induce new blood vessel formation in ischemic disease associated with myocardial infarction, stroke and diabetes. Another potential application is in the field of tissue engineering where it can be used to prevascularize tissues. The identification of this factor will allow the development of in-vitro assays to study the biology of lumenized angiogenesis and to screen compounds for their antiangiogenic activity.

Accordingly, in one aspect, the present invention provides compositions and methods for modulating blood vessel. In one embodiment, a method for modulating a blood vessel in a subject in need thereof comprises contacting a cell of the subject with a procollagen carboxy-terminal propeptide, a biologically active fragment or mimetic thereof, thereby modulating the blood vessel. The method can further comprise contacting a cell of the subject with one or more endothelial growth factors. In a specific embodiment, the one or more endothelial growth factors is vascular endothelial growth factor.

In certain embodiments, the method increases or decreases blood vessel formation relative to an untreated control tissue or organ. In particular embodiments, the method stabilizes or remodels a blood vessel in a tissue or organ relative to an untreated control tissue or organ. In a specific embodiment, the procollagen c-terminal propeptide is selected from the group consisting of collagen I, collagen II, collagen III, collagen V, collagen XI, collagen XXIV, and collagen XXVII. In a more specific embodiment, the procollagen c-terminal propeptide is collagen I.

The present invention also provides a method for decreasing angiogenesis in a subject in need thereof comprising contacting a cell of the subject with an agent that inhibits the expression or biological activity of a procollagen carboxy-terminal propeptide. In one embodiment, the subject has a disease, disorder, or tissue damage and the contacting step ameliorates the disease, disorder, or tissue damage. In another embodiment, a method of treating pathological neovascularization in a subject comprises administering to the subject an agent that decreases angiogenesis in the subject, thereby treating pathological neovascularization in the subject. In such embodiments, the method decreases angiogenesis in a tissue or organ of the subject by at least 5% compared to an untreated control tissue or organ.

In a specific embodiment, the tissue is a neoplastic tissue. In certain embodiments, the cell, tissue or organ can be selected from the group consisting of brain, nervous tissue, eye, ocular tissue, heart, cardiac tissue, and skeletal muscle tissue bladder, bone, brain, breast, cartilage, nervous tissue, esophagus, fallopian tube, heart, pancreas, intestines, gallbladder, kidney, liver, lung, ovaries, prostate, skeletal muscle, skin, spinal cord, spleen, stomach, testes, thymus, thyroid, trachea, urogenital tract, ureter, urethra, and uterus.

In other embodiments, the agent is an antibody or an aptamer that binds a procollagen c-terminal propeptide. In another embodiment, the agent is an inhibitory nucleic acid molecule that decreases the expression of a procollagen c-terminal propeptide. More specifically, the inhibitory nucleic acid molecule is an antisense oligonucleotide, a short interfering RNA (siRNA), or a short hairpin RNA (shRNA). In the methods described herein, the subject can be a human.

The present invention also provides a method for increasing blood vessel formation in a tissue or organ comprising contacting a cell of the tissue or organ with a procollagen c-terminal propeptide, biologically active fragment or mimetic thereof thereby increasing blood vessel formation in the tissue or organ. In another embodiment, a method for stabilizing a blood vessel in a tissue or organ comprises contacting a cell of the tissue or organ with a procollagen c-terminal propeptide, biologically active fragment, or mimetic thereof, thereby stabilizing a blood vessel in the subject. In another embodiment, a method for increasing blood vessel formation or stabilizing or remodeling a blood vessel in a tissue or organ comprises contacting a cell of the tissue or organ with a nucleic acid molecule encoding a procollagen c-terminal propeptide, biologically active fragment, or mimetic thereof, thereby increasing blood vessel formation or stabilizing or remodeling a blood vessel in a tissue or organ. In such embodiment, the contacting increases blood vessel formation or stabilizes a blood vessel in a tissue or organ of a subject. In a more particular embodiment, the tissue or organ is selected from the group consisting of bladder, bone, breast, cartilage, esophagus, fallopian tube, pancreas, intestines, gallbladder, kidney, liver, lung, ovaries, prostate, skin, spinal cord, spleen, stomach, testes, thymus, thyroid, trachea, urogenital tract, ureter, urethra, uterus, brain, nervous tissue, eye, ocular tissue, heart, cardiac tissue, and skeletal muscle tissue.

In certain embodiments, the contacting occurs in vitro or in vivo. In another embodiment, the cell is a human cell. In particular, the cell is an endothelial cell, pericyte, muscle cell, neuron or a glial cell. In the present invention, the cell is present in a subject that has a disease, disorder, or tissue damage and the contacting ameliorates the disease, disorder, or tissue damage.

In another aspect, the present invention provides inhibitory nucleic acid molecules. In one embodiment, the present invention provides an inhibitory nucleic acid molecule that specifically binds at least a fragment of a nucleic acid molecule encoding a procollagen c-terminal propeptide and decreases the expression of the procollagen c-terminal propeptide. The inhibitory nucleic acid molecule can be an siRNA, an antisense oligonucleotide, an shRNA, or a ribozyme.

In yet another aspect, the present invention provides apatamers. In one embodiment, the present invention provides an aptamer that specifically binds at least a fragment of a procollagen c-terminal propeptide and decreases a biological activity of the procollagen c-terminal propeptide.

In other embodiment, the present invention provides a vector comprising a nucleic acid molecule encoding a procollagen c-terminal propeptide, biologically active fragment or mimetic thereof, or encoding an inhibitory nucleic acid molecule described herein, wherein the nucleic acid molecule is positioned for expression. In a specific embodiment, the nucleic acid molecule is operably linked to a promoter suitable for expression in a mammalian cell. In another embodiment, a host cell can comprise a nucleic acid molecule described herein. In a specific embodiment, the host cell is a human cell. In another embodiment, the cell is in vitro or in vivo.

In yet another aspect, the present invention provides pharmaceutical compositions. In a specific embodiment, a pharmaceutical composition for modulating a blood vessel in a subject comprises an effective amount of a procollagen c-terminal propeptide, biologically active fragment or mimetic thereof in a pharmaceutically acceptable excipient. In another embodiment, a pharmaceutical composition for modulating a blood vessel in a subject comprises an effective amount of an inhibitory nucleic acid molecule that reduces the expression of a procollagen c-terminal propeptide in a pharmaceutically acceptable excipient.

In a further embodiment, a pharmaceutical composition for modulating a blood vessel in a subject comprises an effective amount of an aptamer that specifically binds a procollagen c-terminal propeptide or biologically active fragment thereof in a pharmaceutically acceptable excipient. In an alternative embodiment, a pharmaceutical composition for modulating a blood vessel in a subject comprises an effective amount of an antibody that specifically binds a procollagen c-terminal propeptide or biologically active fragment thereof in a pharmaceutically acceptable excipient. In yet another embodiment, a pharmaceutical composition comprises an effective amount of a vector comprising a nucleic acid molecule encoding a procollagen c-terminal propeptide or biologically active fragment in a pharmaceutically acceptable excipient, wherein expression of the propeptide in a cell is capable of modulating a blood vessel.

In another aspect, the present invention provides kits. In a specific embodiment, a kit for modulating blood vessel formation in a subject in need thereof comprises an effective amount of a procollagen c-terminal propeptide or biological fragment thereof and directions for the use of the propeptide for modulating a blood vessel. In another embodiment, a kit for modulating blood vessel formation in a subject in need thereof comprises an effective amount of a nucleic acid molecule encoding a procollagen c-terminal propeptide or biological fragment thereof and directions for the use of the nucleic acid molecule for modulating a blood vessel formation

A kit for decreasing angiogenesis in a subject in need thereof may comprise an effective amount of an aptamer that specifically binds a procollagen c-terminal propeptide or biologically active fragment thereof and directions for the use of the aptamer to decrease angiogenesis in a subject. In another embodiment, a kit for decreasing angiogenesis in a subject in need thereof comprises an effective amount of an antibody that specifically binds a procollagen c-terminal propeptide or biologically active fragment thereof and directions for the use of the antibody to decrease angiogenesis in a subject.

The present invention also provides screening methods involving a procollagen c-terminal propeptide. In a specific embodiment, a method of identifying a compound that modulates blood vessel formation comprises contacting a cell that expresses a procollagen c-terminal propeptide nucleic acid molecule with a candidate compound, and comparing the level of expression of the nucleic acid molecule in the cell contacted by the candidate compound with the level of expression in a control cell not contacted by the candidate compound, wherein an alteration in expression of the procollagen c-terminal propeptide nucleic acid molecule identifies the candidate compound as a compound that modulates blood vessel formation. In another embodiment, a method of identifying a compound that modulates blood vessel formation comprises contacting a cell that expresses a procollagen c-terminal propeptide with a candidate compound, and comparing the level of expression of the propeptide in the cell contacted by the candidate compound with the level of propeptide expression in a control cell not contacted by the candidate compound, wherein an alteration in the expression of the procollagen c-terminal propeptide identifies the candidate compound as a compound that modulates blood vessel formation.

In yet another embodiment, a method of identifying a compound that modulates blood vessel formation comprises contacting a cell that expresses a procollagen c-terminal propeptide with a candidate compound, and comparing the biological activity of the propeptide in the cell contacted by the candidate compound with the level of biological activity in a control cell not contacted by the candidate compound, wherein an alteration in the biological activity of the procollagen c-terminal propeptide identifies the candidate compound as a candidate compound that modulates blood vessel formation. In a specific embodiment, the cell is in vitro. In another embodiment, the cell is in vivo. In other embodiments, the cell is a human cell. In particular embodiments, the cell is an endothelial cell. In a specific embodiment, the cell is a human umbilical vein endothelial cell (HUVEC). In an alternative embodiment, the cell is a human embryonic kidney 293s cell (HEK293s). In yet another embodiment, the screening methods comprise measuring tube formation in the cell. In particular embodiments, the alteration in expression is assayed using an immunological assay, an enzymatic assay, or a radioimmunoassay.

In yet another embodiment, the present invention provides a method for identifying a compound that modulates blood vessel formation comprising (a) providing an assay system comprising a procollagen c-terminal propeptide; (b) contacting the assay system with a test agent under conditions whereby, but for the presence of the test agent, the system provides a reference activity; and (c) detecting a test agent-biased activity of the assay system, wherein a difference between the test agent-biased activity and the reference activity identifies the test agent as a candidate blood vessel formation modulating agent. The assay system can include a screening assay comprising a procollagen c-terminal propeptide and the candidate test agent is a small molecule modulator. Alternatively, the assay system includes a binding assay comprising a procollagen c-terminal propeptide and the candidate test agent is an antibody. In another embodiment, the assay system comprises cultured cells or a non-human animal expressing procollagen c-terminal propeptide. In a specific embodiment, the assay system comprises cultured cells.

In certain embodiments, the assay detects an event selected from the group consisting of cell proliferation, cell cycling, apoptosis, tubulogenesis, cell migration, cell sprouting and response to hypoxic conditions. In a specific embodiment, the assay detects tubulogenesis or cell migration or cell sprouting. In a more specific embodiment, the assay detects cell sprouting. In yet another embodiment, the assay system comprises the step of testing the cellular response to stimulation with one or more proangiogenic agents.

The present invention also provides a method for prevascularizing a tissue graft comprising contacting a cell of the tissue with a procollagen carboxy-terminal propeptide, a biologically active fragment or mimetic thereof, thereby prevascularizing the tissue graft. The method can further comprise contacting a cell of the subject with one or more endothelial growth factors. In a specific embodiment, the one or more endothelial growth factors is vascular endothelial growth factor.

In the methods described herein, the procollagen c-terminal propeptide is selected from the group consisting of collagen I, collagen II, collagen III, collagen V, collagen XI, collagen XXIV, and collagen XXVII. In a specific embodiment, the procollagen c-terminal propeptide is collagen I.

### BRIEF DESCRIPTION OF THE FIGURES

FIG. 1 shows the strategy adopted to isolate stromal growth factors.

FIG. 2 is a chart indicating the relative activity of low dose VEGF plus conditioned media fractions.

FIG. 3 is a chart indicating the relative activity of high dose VEGF plus conditioned media fractions, revealing the activity of PICP.

FIG. 4 shows the mass spec results for PICP peptide fragments.

FIG. 5 is a schematic showing processing of procollagen.

FIG. 6 shows the results of the spheroid sprouting assay using procollagen I c-terminal propeptide (PICP). This assay depends on stromal cell support to allow for the generation of capillary like structures. Endothelial cells are seeded onto a dextran bead and then embedded into a matrix such a fibrin. Conditioned media from lung fibroblasts, which contains PICP and high concentrations of vascular endothelial growth factor (VEGF), is concentrated and added to the assay as a positive control. The PICP fragment (amino acids 1219-1464) was cloned into a lentiviral expression vector (Clontech). Lentiviral particles were generated using a standard technique and HEK 293F cells were transduced and selected with puromycin for stable protein expression. A fusion protein was secreted into the media. The media containing the fusion protein (PICP) was able to induce lumenized sprouting even more prominently than the positive control. VEGF by itself, even at high doses, is unable to induce sprouting.

FIG. 7 demonstrates that an N1365A mutation of PICP results not only in lost function but also acts as a competitive inhibitor.

FIG. 8 shows that PICP has a direct effect on prostate cancer cells in vitro. FIG. 8A is a negative control showing 24-hour growth of the prostate cancer cell line DU145. In FIG. 8B, significantly more growth of DU145 in the presence of PICP is seen over 24 hours.

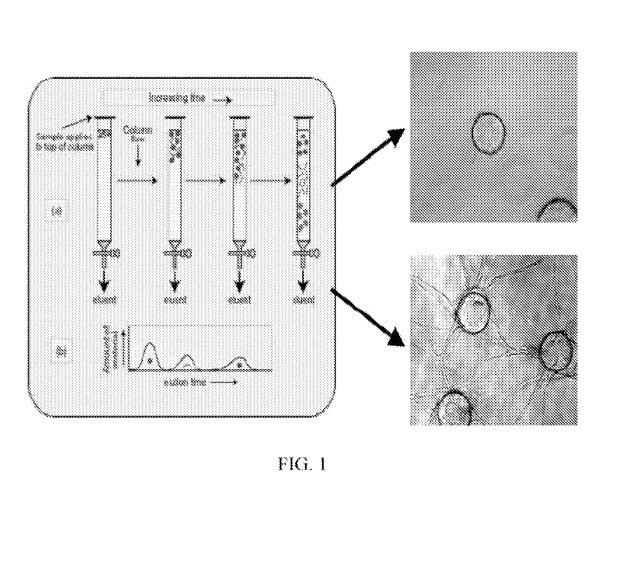

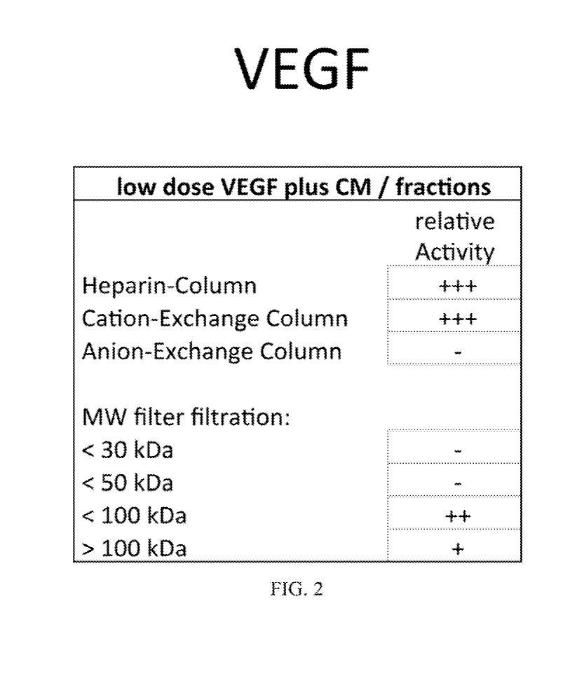

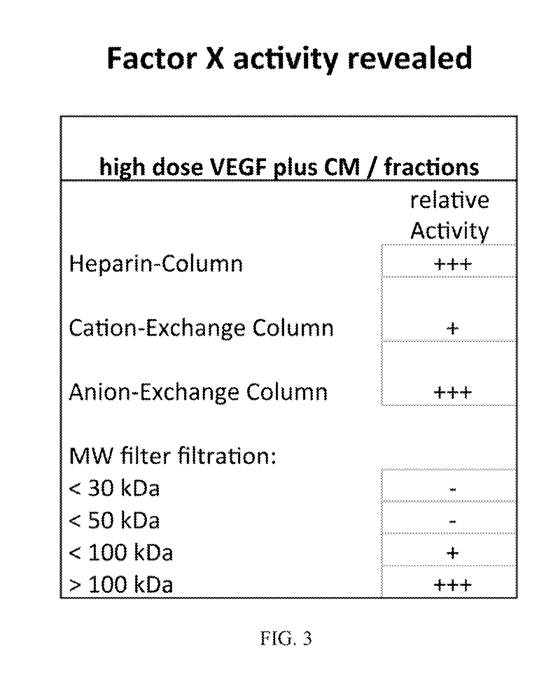

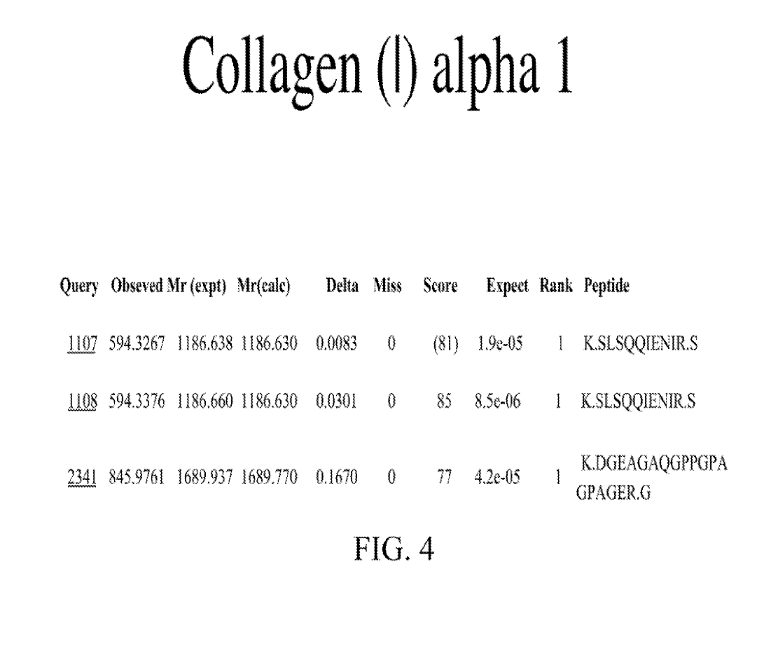

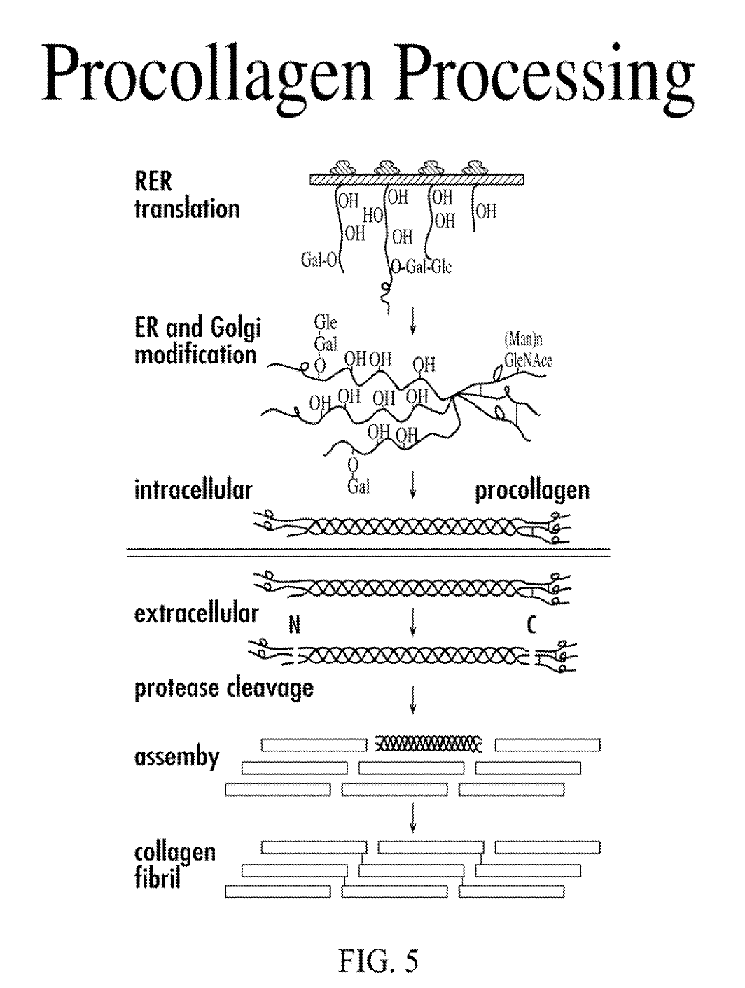

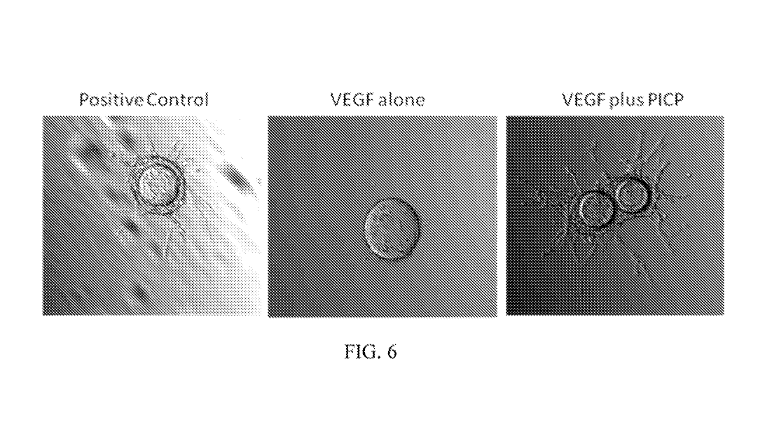

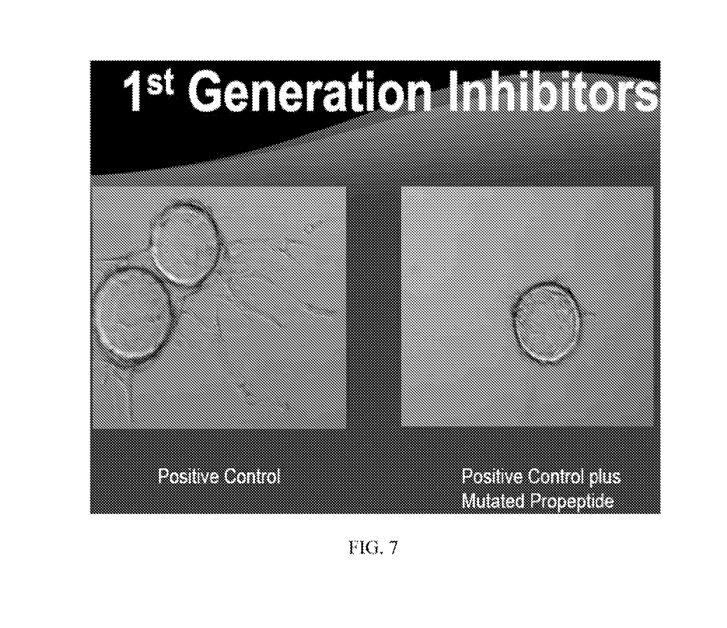

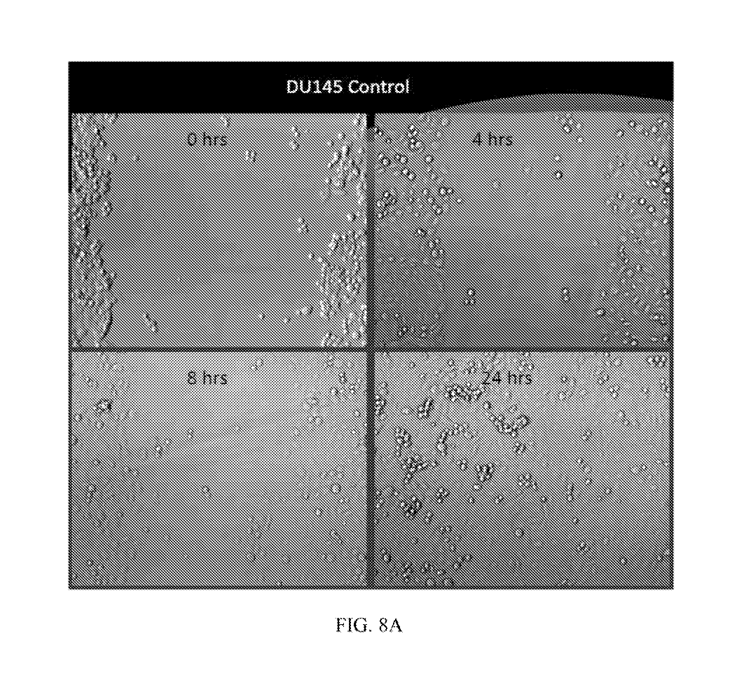

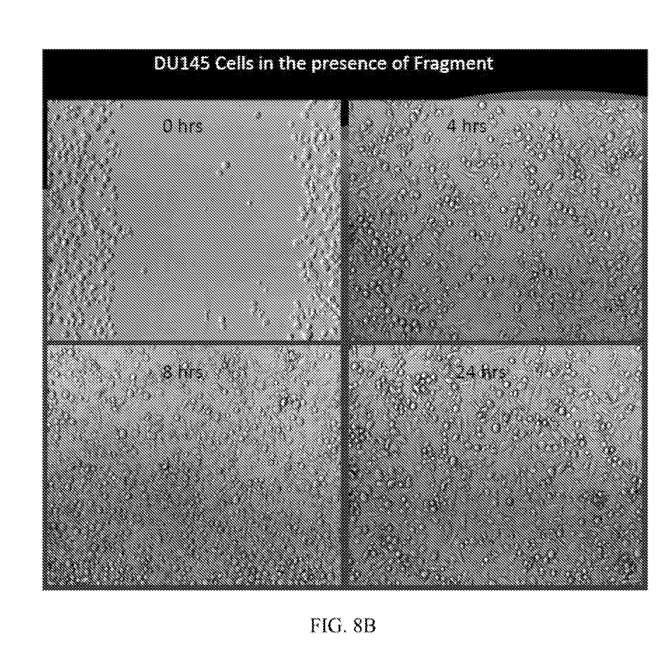

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

To identify novel targets and proangiogenic molecules, the present inventors took inspiration from the most advanced in-vitro assays of angiogenesis. In vitro assays of angiogenesis display features of prolonged stability of vascular structures and are typically lumenenized. Interestingly, these assays generally combine endothelial cell growth factors and the co-culture with fibroblasts or other mesenchymal cells. One of the earliest assays was devised almost twenty years ago, and several variants on the concept were developed. The assay used to screen for the biological activity of fractions obtained from the conditioned media of fibroblasts is based on a sprouting assay. Briefly, endothelial cells are seeded onto dextran beads, embedded into a fibrin matrix and media containing VEGF as well as fibroblasts or fibroblast conditioned media was seeded on top. Over about 7-10 days, capillary-like structures start to invade the matrix displaying the characteristics of tip cells, a stalk and lumen formation. In the presence of just growth factors, no lumenized structures will form. Until the present invention, the exact mechanism of how fibroblasts or other mesenchymal cells can facilitate this effect has not been known. Understanding the exact mechanism how activated stroma such as fibroblasts can support blood vessels would have widespread applications. Tissue remodeling, i.e., stroma activation, occurs with virtually any type of injury, wound healing, new blood vessel formation or tumor growth. Furthermore, there is supporting animal data that stromal cells are required to support long-lasting vasculature in engineered tissues. See Au et al., 111(9) BLOOD 4551-58 (2008); and Koike et al., 428(6979) NATURE 138-9 (2004).

To identify the protein responsible for this effect, the conditioned media from lung fibroblasts was fractionated, tested in the sprouting assay, and positive fractions were sent to the proteomics core for analysis via mass spectrometry. One of the two key insights made by the present inventors that lead to the discovery of the protein, was that relatively high concentrations of recombinant VEGF were necessary to fully substitute for the conditioned media. This means that the conditioned media and some of its fractions contained both VEGF and the unknown protein. Once the screening assay was substituted with high doses of VEGF (on the equivalent of 500 ng/ml—the protein was likely sequestered in the matrix), the present inventors were able to track down the unknown protein.

One of the proteins which the present inventors had ignored for most of the time was collagen I because collagen I matrix per se does not support vascular structures. However, when a closer look was taken at the peptides seen on the mass spec, the vast majority of them were derived from the c-terminal part of the precursor molecule of collagen I, the procollagen I. During collagen I synthesis, the heterotrimeric procollagen I molecule (made of 2×col 1a1 and 1×col 1a2) assembles within the cells and after secretion, the c-terminal propeptide (PICP) is cleaved off and is not part of the mature collagen fibril which is present in collagen gels. The critical and novel role in the ability of PICP to induce stable, lumenized capillary like structures in relevant angiogenesis models has not been described to date. In fact, the present inventors believe that PICP and its relatives (the c-terminal propeptides of collagen II, III, V, XI, XXIV and XXVII) partake in what has been coined as the angiogenic switch. This means that whenever, there is tissue injury, fibroblasts start to repair the area with collagen I, which is a ubiquitous protein, and at the same generate PICP which facilitates blood vessel formation. In addition, when fibroblasts cease to repair and remodel, PICP production ceases and blood vessel formation is turn off.

To prove that targeting or modifying a procollagen c-terminal propeptide molecule, e.g., PICP, can suppress fibroblast supported angiogenesis, a mutant PICP was generated. PICP contains a highly conserved glycosylation site (amino acid 1365)—the function of which is unknown. When the amino acid was mutated from an asparagine to an alanine, the protein not only lost its function but also acted as a potential competitive inhibitor. This would be a first generation inhibition, which is a proof of principle that targeting this process can have widespread application in conditions of pathological angiogenesis. Accordingly, the proangiogenic properties of PICP could be used alone or in combination in areas of ischemic disease, wound healing, tissue regeneration, burn wounds, tissue engineering.

### I. DEFINITIONS

A “procollagen carboxy-terminal propeptide” is a protein or protein variant or fragment thereof, that is substantially identical to at least a portion of a procollagen c-terminal propeptide and that has a procollagen c-terminal propeptide biological activity (e.g., modulating angiogenesis, vasculogenesis, blood vessel remodeling, regression, or persistence). Example of procollagen c-terminal propeptides include collagen I (SEQ ID NO:1), collagen II (SEQ ID NO:11), collagen III (SEQ ID NO:3), collagen V (SEQ ID NO:5), collagen XI (SEQ ID NO:7), collagen XXIV (SEQ ID NO:12) and collagen XVIII (SEQ ID NO:9). See Ricard-Blum, S., COLD SPRING HARB. PERSPECT. BIOL. Doi 10.1101/cshperspect.a004978.

By “PICP polypeptide” is meant a protein or protein variant, or fragment thereof, that is substantially identical to at least a portion of a procollagen (I) c-terminal propeptide polypeptide and that has a PICP biological activity (e.g., modulating angiogenesis, vasculogenesis, blood vessel remodeling, regression, or persistence).

By “PIICP polypeptide” is meant a protein or protein variant, or fragment thereof, that is substantially identical to at least a portion of a procollagen (II) c-terminal propeptide polypeptide and that has a PICP biological activity (e.g., modulating angiogenesis, vasculogenesis, blood vessel remodeling, regression, or persistence).

By “PIIICP polypeptide” is meant a protein or protein variant, or fragment thereof, that is substantially identical to at least a portion of a procollagen (III) c-terminal propeptide polypeptide and that has a PICP biological activity (e.g., modulating angiogenesis, vasculogenesis, blood vessel remodeling, regression, or persistence).

By “PVCP polypeptide” is meant a protein or protein variant, or fragment thereof, that is substantially identical to at least a portion of a procollagen (V) c-terminal propeptide polypeptide and that has a PICP biological activity (e.g., modulating angiogenesis, vasculogenesis, blood vessel remodeling, regression, or persistence).

By “PXICP polypeptide” is meant a protein or protein variant, or fragment thereof, that is substantially identical to at least a portion of a procollagen (XI) c-terminal propeptide polypeptide and that has a PICP biological activity (e.g., modulating angiogenesis, vasculogenesis, blood vessel remodeling, regression, or persistence).

By “PXXIVCP polypeptide” is meant a protein or protein variant, or fragment thereof, that is substantially identical to at least a portion of a procollagen (XXIV) c-terminal propeptide polypeptide and that has a PICP biological activity (e.g., modulating angiogenesis, vasculogenesis, blood vessel remodeling, regression, or persistence).

By “PXVIICP polypeptide” is meant a protein or protein variant, or fragment thereof, that is substantially identical to at least a portion of a procollagen (XVII) c-terminal propeptide polypeptide and that has a PICP biological activity (e.g., modulating angiogenesis, vasculogenesis, blood vessel remodeling, regression, or persistence).

A “procollagen c-terminal propeptide nucleic acid molecule” refers to a polynucleotide encoding a procollagen c-terminal propeptide (e.g., PICP) or variant, or fragment thereof.

The term “procollagen c-terminal propeptide biological activity” means any effect on the vasculature. Specifically, procollagen c-terminal propeptide biological activities include, but are not limited to, increasing or decreasing blood vessel formation, blood vessel stabilization, regression, or persistence, modulation of blood vessel remodeling, or procollagen c-terminal propeptide antibody binding.

An “agent” is a compound, polynucleotide, or polypeptide that modulates the expression or biological activity of a target gene or polypeptide.

By “ameliorate” is meant decrease, suppress, attenuate, diminish, arrest, or stabilize the development or progression of a disease.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and O-phosphoserine, phosphothreonine.

An “amino acid analog” refers to a compound that has the same basic chemical structure as a naturally occurring amino acid, i.e., a carbon that is bound to a hydrogen, a carboxyl group, an amino group, and an R group (e.g., homoserine, norleucine, methionine sulfoxide, methionine methyl sulfonium), but that contains some alteration not found in a naturally occurring amino acid (e.g., a modified side chain). The term “amino acid mimetic” refers to chemical compounds that have a structure that is different from the general chemical structure of an amino acid, but that function in a manner similar to a naturally occurring amino acid. Amino acid analogs may have modified R groups (for example, norleucine) or modified peptide backbones, but retain the same basic chemical structure as a naturally occurring amino acid. In one embodiment, an amino acid analog is a D-amino acid, a beta-amino acid, or an N-methyl amino acid.

Amino acids and analogs are well known in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “angiogenesis” refers to the growth of new blood vessels originating from existing blood vessels. Angiogenesis can be assayed by any number of methods known to those of ordinary skill in the art including, but not limited to, measuring the number of non-branching blood vessel segments (number of segments per unit area), the functional vascular density (total length of perfused blood vessel per unit area), the vessel diameter, or the vessel volume density (total of calculated blood vessel volume based on length and diameter of each segment per unit area).

By “antibody” is meant any immunoglobulin polypeptide, or fragment thereof, having immunogen binding ability.

An “aptamer” is an oligonucleotide that binds to a protein.

The term “blood vessel formation” refers to the dynamic process that includes one or more steps of blood vessel development and/or maturation. Methods for measuring blood vessel formation and maturation are standard in the art and are described, for example, in Jain et al. 2 NAT. REV. CANCER 266-76 (2002).

The term “blood vessel remodeling” refers to the structural remodeling and/or differentiation of a blood vessel network. In one embodiment, remodeling alters intimal hyperplasia. In another embodiment, remodeling supports the maturation of an immature blood vessel network. In some embodiments, blood vessel maturation includes the elimination of extraneous vessels.

By “an effective amount” is meant the amount of a required to ameliorate the symptoms of a disease relative to an untreated patient. The effective amount of active compound(s) used to practice the present invention for therapeutic treatment of a vascular disease varies depending upon the manner of administration, the age, body weight, and general health of the subject. Ultimately, the attending physician or veterinarian will decide the appropriate amount and dosage regimen. Such amount is referred to as an “effective” amount.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

By “fragment” is meant a portion (e.g., at least about 5, 10, 25, 50, 100, 125, 150, 200, 250, 300, 350, 400, or 500 amino acids or nucleic acids) of a protein or nucleic acid molecule that is substantially identical to a reference protein or nucleic acid and retains at least one biological activity of the reference. In some embodiments the portion retains at least 50%, 75%, or 80%, or more preferably 90%, 95%, or even 99% of the biological activity of the reference protein or nucleic acid described herein.

A “host cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

By “inhibitory nucleic acid” is meant a double-stranded RNA, siRNA (short interfering RNA), shRNA (short hairpin RNA), or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule.

The terms “isolated,” “purified,” or “biologically pure” refer to material that is free to varying degrees from components which normally accompany it as found in its native state. Various levels of purity may be applied as needed according to this invention in the different methodologies set forth herein; the customary purity standards known in the art may be used if no standard is otherwise specified.

By “isolated nucleic acid molecule” is meant a nucleic acid (e.g., a DNA, RNA, or analog thereof) that is free of the genes which, in the naturally occurring genome of the organism from which the nucleic acid molecule of the present invention is derived, flank the gene. The term therefore includes, for example, a recombinant DNA that is incorporated into a vector, into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “modulation” is meant a change (increase or decrease) in the expression level or biological activity of a gene or polypeptide as detected by standard methods known in the art. As used herein, modulation includes at least about 10% change, 25%, 40%, 50% or a greater change in expression levels or biological activity (e.g., about 75%, 85%, 95% or more).

The term “mimetic” means an agent having a structure that is different from the general chemical structure of a reference agent, but that has at least one biological function of the reference.

By “modulating a blood vessel” is meant altering angiogenesis, vasculogenesis, blood vessel stabilization, regression, persistence, or remodeling.

The term “nucleic acid” refers to an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

Specific examples of some nucleic acids envisioned for this invention may contain phosphorothioates, phosphotriesters, methyl phosphonates, short chain alkyl or cycloalkyl intersugar linkages or short chain heteroatomic or heterocyclic intersugar linkages. Also preferred are oligonucleotides having morpholino backbone structures (Summerton, J. E. and Weller, D. D., U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen et al. Science 199: 254, 1997). Other preferred oligonucleotides may contain alkyl and halogen-substituted sugar moieties comprising one of the following at the 2′ position: OH, SH, SCH₃, F, OCN, O(CH₂)ₙNH₂ or O(CH₂)ₙCH₃, where n is from 1 to about 10; C₁ to C₁₀ lower alkyl, substituted lower alkyl, alkaryl or aralkyl; Cl; Br; CN; CF₃; OCF₃; O—, S—, or N-alkyl; O—, S—, or N-alkenyl; SOCH₃; SO₂CH₃; ONO₂; NO₂; N3; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group. Other preferred embodiments may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines.

The term “operably linked” means that a first polynucleotide is positioned adjacent to a second polynucleotide that directs transcription of the first polynucleotide when appropriate molecules (e.g., transcriptional activator proteins) are bound to the second polynucleotide.

By “pathological neovascularization” is meant an excess or abnormal formation of blood vessels in a tissue or organ.

By “recombinant” is meant the product of genetic engineering or chemical synthesis. By “positioned for expression” is meant that the polynucleotide of the present invention (e.g., a DNA molecule) is positioned adjacent to a DNA sequence that directs transcription and translation of the sequence (i.e., facilitates the production of, for example, a recombinant protein of the present invention, or an RNA molecule).

The term “reference” means a standard or control condition.

By “ribozyme” is meant an RNA that has enzymatic activity, possessing site specificity and cleavage capability for a target RNA molecule. Ribozymes can be used to decrease expression of a polypeptide. Methods for using ribozymes to decrease polypeptide expression are described, for example, by Turner et al., (Adv. Exp. Med. Biol. 465:303-318, 2000) and Norris et al., (Adv. Exp. Med. Biol. 465:293-301, 2000).

By “siRNA” is meant a double stranded RNA. Optimally, an siRNA is about 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to down-regulate mRNA levels or promoter activity.

By “specifically binds” is meant a molecule (e.g., peptide, polynucleotide) that recognizes and binds a protein or nucleic acid molecule of the present invention, but which does not substantially recognize and bind other molecules in a sample, for example, a biological sample, which naturally includes a protein of the present invention.

By “stabilizes” a blood vessel is meant increases the survival or maintenance of the blood vessel in a tissue relative to a control tissue.

By “subject” is meant a mammal, including, but not limited to, a human or non-human mammal, such as a bovine, equine, canine, ovine, or feline.

By “substantially identical” is meant a protein or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e.sup.-3 and e.sup.-100 indicating a closely related sequence.

By “transformed cell” is meant a cell into which (or into an ancestor of which) has been introduced, by means of recombinant DNA techniques, a polynucleotide molecule encoding (as used herein) a protein of the present invention.

By “vascular disease or disorder” is meant any pathology that disrupts the normal function of a blood vessel or that results in excess or abnormal blood vessel formation. Exemplary vascular diseases or disorders include, but are not limited to, atherosclerosis, restenosis, systemic and pulmonary hypertension, intimal hyperplasia, peripheral artery disease, limb ischemia, cancer, arthritis, cardiac ischemia, age related macular degeneration, and stroke.

By “vasculogenesis” is meant the development of new blood vessels originating from stem cells, angioblasts, or other precursor cells.

Accordingly, the present invention features compositions and methods that are useful for modulating angiogenesis, vasculogenesis, blood vessel stabilization, regression, persistence, or remodeling. As reported in more detail below, the present invention is based, at least in part, on the discovery that procollagen c-terminal propeptide modulates blood vessel formation and function.

### II. PROCOLLAGEN CARBOXY-TERMINAL PROPEPTIDE

As described herein, the present invention provides compositions and methods useful for modulating angiogenesis. In one aspect, the present invention involves the use of a procollagen carboxy-terminal propeptide.

In one aspect, the procollagen carboxy-terminal propeptide is procollagen (I) c-terminal propeptide. See amino acids 1219-1464 of SEQ ID NO:1, and SEQ ID NO:2. Type I collagen is the most abundant collagen species in many soft tissues and accounts for more than 90% of the organic matrix of mineralized bone. It is synthesized in the form of a larger protein, type I procollagen, which contains relatively long additional sequences at both ends. These sequences, known as the N- and C-terminal propeptides of type I procollagen, are removed by two specific proteinases in the extracellular space. Proper cleavage of the precursor-specific parts of the molecule is a prerequisite for the appropriate assembly of type I collagen molecules into collagen fibers. The C-terminal propeptide of type I procollagen (PICP), when cleaved off intact from the procollagen molecule, is found in free form interstitial fluid, e.g., in healing wounds and also in blood, where its concentration is thought to reflect type I collagen synthesis in the body.

Similarly, c-terminal propeptides from other collagen types can be used. For example, c-terminal propeptides from collagen III (amino acids 154-1221 of SEQ ID NO:3, and SEQ ID NO:4), collagen V (amino acids 1605-1838 of SEQ ID NO:5, and SEQ ID NO:6), collagen XI (amino acids 1564-1806 of SEQ ID NO:7, and SEQ ID NO:8), collagen XXVII (amino acids 625-1621 of SEQ ID NO:9, and SEQ ID NO:10), collagen II (SEQ ID NO:11) and collagen XXIV (SEQ ID NO:12).

### III. PATHOLOGICAL NEOVASCULARIZATION

The modulation of procollagen c-terminal propeptide expression or biological activity is likely to be broadly useful for the treatment or prevention of diseases or disorders that can be ameliorated by the modulation of angiogenesis, or blood vessel remodeling or stabilization. Diseases and disorders susceptible to treatment by the modulation of procollagen c-terminal propeptide expression or biological activity include those characterized by abnormal, diminished or excess blood vessel formation including, but not limited to, pathological neovascular disorders; blood vessels to solid tumors or neoplasia; vascular malformations both benign and malignant; vascular abnormalities in development, such as hemangiomas, vascular malformations or the failure to develop normal structures related to abnormal blood vessel development. Disorders characterized by the absence of vessel formation include birth defects, vascular insufficiency, and failure to develop collateral blood vessels in response to stress, such as ischemia. Examples include, but are not limited to, peripheral vascular or coronary vascular disease disorders that require an alteration in vascular remodeling, including cancer, arthritis, atherosclerosis, restenosis after angioplasty, systemic and pulmonary hypertension, atherosclerosis, embryonic or fetal development, or vascular response to common or atypical disease. In particular diseases such as restenosis, the remodeling process involves endothelial cell injury and/or dysfunction that results in intimal/medial thickening. In addition, the present invention provides methods and compositions for the treatment of diseases or disorders that require an increase in blood vessel formation (e.g., peripheral artery disease, limb ischemia, cardiac ischemia, and stroke.

### IV. THERAPEUTIC METHODS

The present invention provides methods of treating a vascular disease, disorder or symptom thereof that can be ameliorated by the modulation of angiogenesis, vasculogenesis, blood vessel stabilization, regression, persistence, or remodeling. The methods comprise administering a therapeutically effective amount of a pharmaceutical composition comprising an agent described herein (e.g., an agent that increases or decreases procollagen c-terminal propeptide expression or biological activity) to a subject (e.g., a mammal such as a human). Thus, in one embodiment, the present invention features a method of treating a subject suffering from or susceptible to a disease or disorder or symptom thereof that requires an increase in blood vessel formation or stabilization. Alternatively, the present invention provides compositions and methods for reducing pathological neovascularization. The method includes the step of administering to the mammal a therapeutic amount of an agent described herein sufficient to treat the vascular disease or disorder or symptom thereof, under conditions such that the disease or disorder is treated.

The methods herein include administering to the subject (including a subject identified as in need of such treatment) an effective amount of an agent described herein, or a composition described herein to produce such effect. Identifying a subject in need of such treatment can be in the judgment of a subject or a health care professional and can be subjective (e.g., opinion) or objective (e.g., measurable by a test or diagnostic method).

The therapeutic methods of the present invention, which include prophylactic treatment, in general comprise administration of a therapeutically effective amount of the agents herein, such as a compound to a subject (e.g., animal, human) in need thereof, including a mammal, particularly a human. Such treatment will be suitably administered to subjects, particularly humans, suffering from, having, susceptible to, or at risk for a vascular disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like). The compounds herein may be also used in the treatment of any other vascular disorders in which modulation of angiogenesis is required or in which pathological neovascularization may be implicated.

### V. POLYNUCLEOTIDES ENCODING PROCOLLAGEN C-TERMINAL PROPEPTIDE

In general, the present invention features the use of nucleic acid sequences that encode a procollagen c-terminal propeptide or biologically active fragment thereof sufficient to modulate angiogenesis, vasculogenesis, blood vessel remodeling, or blood vessel stabilization. Also included in the methods of the present invention are nucleic acid molecules containing at least one strand that hybridizes with a procollagen c-terminal propeptide nucleic acid sequence (e.g., inhibitory nucleic acid molecules that reduce procollagen c-terminal propeptide expression, such as a dsRNA, siRNA, shRNA, or antisense oligonucleotides, microRNA, ribozymes, aptamers, monoclonal antibodies or other). An isolated nucleic acid molecule can be manipulated using recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid molecule that is isolated within a cloning or expression vector may comprise only a tiny percentage of the material in the cell in which it resides. Such a nucleic acid is isolated, however, as the term is used herein, because it can be manipulated using standard techniques known to those of ordinary skill in the art.

### VI. PROCOLLAGEN C-TERMINAL PROPEPTIDE POLYNUCLEOTIDE THERAPY

Polynucleotide therapy featuring a polynucleotide encoding a procollagen c-terminal propeptide, variant, or fragment thereof or encoding an inhibitory nucleic acid molecules that reduce procollagen c-terminal propeptide expression (e.g., a dsRNA, siRNA, shRNA, or antisense oligonucleotides, (microRNA, ribozymes, aptamers, monoclonal antibodies or other) are therapeutic approaches for treating a vascular disease or disorder. Such nucleic acid molecules can be delivered to cells of a subject having a vascular disease or disorder, such as a disease that requires an increase in blood vessel formation or stabilization. The nucleic acid molecules must be delivered to the cells of a subject in a form in which they can be taken up so that therapeutically effective levels of a procollagen c-terminal propeptide or fragment thereof can be produced.

Transducing viral (e.g., retroviral (lentiviral), adenoviral, and adeno-associated viral, herpes viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S. 94:10319, 1997). For example, a polynucleotide encoding a procollagen c-terminal propeptide, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer a procollagen c-terminal propeptide polynucleotide systemically.

Non-viral approaches can also be employed for the introduction of a therapeutic to a cell of a patient diagnosed as having a vascular disease or disorder. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). In some embodiments, the nucleic acids are administered in combination with a liposome and protamine. Administration should be sufficient to modulate angiogenesis, vasculogenesis, blood vessel remodeling, or blood vessel stabilization.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), Chicken Beta Actin (CBA) or metallothionein promoters). Promiscuous, ubiquitous or tissue/cell specific promoters are all useful in the methods of the present invention. The use of such promoters is routine. In other embodiments, promoters encompassed by the present invention are regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

### VII. PROCOLLAGEN C-TERMINAL PROPEPTIDE POLYPEPTIDE THERAPY

Another therapeutic approach included in the present invention involves administration of a recombinant therapeutic, such as a recombinant procollagen c-terminal propeptide, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

In one embodiment, procollagen c-terminal propeptides are expressed in vascular cells, such as an endothelial cells, endothelial progenitor cells, pericytes, or astrocytes to achieve a therapeutic benefit but this specifically does not exclude any cell of the cells of the target tissues or of the support tissues as potential treatment targets.

As reported herein, procollagen c-terminal propeptides have direct effects or effects mediated through relevant pathways on blood vessel formation or remodeling. Accordingly, the present invention provides therapeutic methods for the treatment of vascular diseases that feature procollagen c-terminal propeptides. In one approach, a procollagen c-terminal propeptide is provided directly to a tissue that requires an increase or decrease in angiogenesis, vasculogenesis, blood vessel remodeling, or blood vessel stabilization. Procollagen c-terminal propeptides for use in therapeutic methods of the present invention are provided by methods known in the art including the purification of a procollagen c-terminal propeptide from a biological sample that endogenously produces the polypeptide or the recombinant production of the procollagen c-terminal propeptide.

In general, procollagen c-terminal propeptides, variants, and fragments thereof are produced by transformation of a suitable host cell with all or part of a polypeptide-encoding nucleic acid molecule or fragment thereof in a suitable expression vehicle. Those skilled in the field of molecular biology will understand that any of a wide variety of expression systems may be used to provide the recombinant protein. The precise host cell used is not critical to the present invention. A polypeptide of the present invention may be produced in a prokaryotic host (e.g., E. coli) or in a eukaryotic host (e.g., Sacchamyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the present invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (Novagen, Inc., Madison, Wis). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione 5-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once the recombinant polypeptide of the present invention is expressed, it is isolated, e.g., using affinity chromatography. In one example, an antibody (e.g., produced as described herein) raised against a polypeptide of the present invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the present invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

### VIII. PROCOLLAGEN C-TERMINAL PROPEPTIDES AND ANALOGS

Also included in the present invention are procollagen c-terminal propeptides, variants, or fragments thereof containing at least one alteration relative to a reference sequence. Desirably, such variants, fragments and analogs maintain at least one biological function of a full length procollagen c-terminal propeptide (i.e., the modulation of angiogenesis, vasculogenesis, blood vessel remodeling, or blood vessel stabilization). Altered procollagen c-terminal propeptides include those having certain mutations, deletions, insertions, or post-translational modifications. The present invention further includes analogs of any naturally-occurring polypeptides of the present invention. Analogs can differ from naturally-occurring polypeptides of the present invention by amino acid sequence differences, by post-translational modifications, or by both. Analogs of the present invention will generally exhibit at least 85%, more preferably 90%, and most preferably 95% or even 99% identity with all or part of a naturally-occurring amino acid sequence of the present invention. The length of sequence comparison is at least 10, 13, 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the present invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids.

In addition to full-length polypeptides, the present invention also includes fragments of any one of the polypeptides of the present invention. As used herein, the term “a fragment” means at least 5, 10, 13, or 15 amino acids. In other embodiments a fragment is at least 20 contiguous amino acids, at least 30 contiguous amino acids, or at least 50 contiguous amino acids, and in other embodiments at least 60 to 80 or more contiguous amino acids. Fragments of the present invention can be generated by methods known to those skilled in the art or may result from normal protein processing (e.g., removal of amino acids from the nascent polypeptide that are not required for biological activity or removal of amino acids by alternative mRNA splicing or alternative protein processing events).

### IX. PROCOLLAGEN C-TERMINAL PROPEPTIDE ANTIBODIES

In another approach, the present invention features methods for reducing angiogenesis, vasculogenesis, blood vessel remodeling, or blood vessel stabilization, for example, by reducing the biological activity of a procollagen c-terminal propeptide. Methods for reducing the biological activity of a procollagen c-terminal propeptide include administering to a subject in need thereof an antibody that specifically binds and disrupts the biological activity of a procollagen c-terminal propeptide. Antibodies are well known to those of ordinary skill in the science of immunology. As used herein, the term “antibody” means not only intact antibody molecules, but also fragments of antibody molecules that retain immunogen binding ability. Such fragments are also well known in the art and are regularly employed both in vitro and in vivo. Accordingly, as used herein, the term “antibody” means not only intact immunoglobulin molecules but also the well-known active fragments F(ab)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983). The antibodies of the present invention comprise whole native anti-bodies, bispecific antibodies; chimeric antibodies; Fab, Fab′, single chain V region fragments (scFv) and fusion polypeptides.

In one embodiment, an antibody that binds a procollagen c-terminal propeptide is monoclonal. Alternatively, the anti-procollagen c-terminal propeptide antibody is a polyclonal antibody. The preparation and use of polyclonal antibodies are also known the skilled artisan. The present invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab.alpha.)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab.alpha. fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing procollagen c-terminal propeptides, or immunogenic fragments thereof; as an immunogen. One method of obtaining antibodies is to immunize suitable host animals with an immunogen and to follow standard procedures for polyclonal or monoclonal anti-body production. The immunogen will facilitate presentation of the immunogen on the cell surface. Immunization of a suitable host can be carried out in a number of ways. Nucleic acid sequences encoding a procollagen c-terminal propeptide, or immunogenic fragments thereof, can be provided to the host in a delivery vehicle that is taken up by immune cells of the host. The cells will in turn express the polypeptide, thereby generating an immunogenic response in the host. Alternatively, nucleic acid sequences encoding a procollagen c-terminal propeptide or immunogenic fragments thereof, can be expressed in cells in vitro, followed by isolation of the polypeptide and administration of the receptor to a suitable host in which antibodies are raised.

Using either approach, antibodies can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Monoclonal antibodies (Mabs) produced by methods of the present invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567; 5,530,101; 5,225,539; 5,585,089; 5,693,762; and 5,859,205.

### X. INHIBITORY NUCLEIC ACIDS

Inhibitory nucleic acid molecules are those oligonucleotides that inhibit the expression or activity of a procollagen c-terminal propeptide. Such oligonucleotides include single and double stranded nucleic acid molecules (e.g., DNA, RNA, and analogs thereof) that bind a nucleic acid molecule that encodes a procollagen c-terminal propeptide (e.g., antisense molecules, siRNA, shRNA, microRNA) as well as nucleic acid molecules that bind directly to a procollagen c-terminal propeptide to modulate its biological activity (e.g., aptamers).

### XI. RIBOZYMES

Catalytic RNA molecules or ribozymes that include an antisense procollagen c-terminal propeptide sequence of the present invention can be used to inhibit expression of a procollagen c-terminal propeptide nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 Al, each of which is incorporated by reference.

Accordingly, the present invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the present invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 bp (desirably 25 to 29 bp), and the loops can range from 4 to 30 by (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### XII. SIRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39. 2002).

Given the sequence of a target gene, siRNAs may be designed to inactivate that gene. Such siRNAs, for example, could be administered directly to an affected tissue, or administered systemically. The nucleic acid sequence of a procollagen c-terminal propeptide gene can be used to design small interfering RNAs (siRNAs). The 21 to 25 nucleotide siRNAs may be used, for example, as therapeutics to treat a vascular disease or disorder.

The inhibitory nucleic acid molecules of the present invention may be employed as double-stranded RNAs for RNA interference (RNAi)-mediated knock-down of procollagen c-terminal propeptide expression. In one embodiment, procollagen c-terminal propeptide expression is reduced in an endothelial cell or an astrocyte. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the present invention, double stranded RNA (dsRNA) molecule is made that includes between eight and nineteen consecutive nucleobases of a nucleobase oligomer of the present invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

### XIII. SHRNAS

Small hairpin RNAs consist of a stem-loop structure with optional 3′ UU-overhangs. While there may be variation, stems can range from 21 to 31 by (desirably 25 to 29 bp), and the loops can range from 4 to 30 by (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### XIV. MICRORNAS

microRNAs (miRNAs) are an abundant class of endogenous non-protein-coding small RNAs, which negatively regulate gene expression at the post-trascriptional level in many developmental and metabolic processes. miRNAs regulate a variety of biological processes, including developmental timing, signal transduction, tissue differentiation and maintenance, disease, and carcinogenesis. MicroRNAs represent a means to down regulate procollagen c-terminal propeptide expression.

### XV. APTAMERS

Nucleic acid aptamers are single-stranded nucleic acid (DNA or RNA) ligands that function by folding into a specific globular structure that dictates binding to target proteins or other molecules with high affinity and specificity, as described by Osborne et al., Curr. Opin. Chem. Biol. 1:5-9, 1997; and Cerchia et al., FEBS Letters 528:12-16, 2002. Desirably, the aptamers are small, approximately 15 KD. The aptamers are isolated from libraries consisting of some 10¹⁴-10′⁵ random oligonucleotide sequences by a procedure termed SELEX (systematic evolution of ligands by exponential enrichment). See Tuerk et al., Science, 249:505-510, 1990; Green et al., Methods Enzymology. 75-86, 1991; Gold et al., Annu. Rev. Biochem., 64: 763-797, 1995; Uphoff et al., Curr. Opin. Struct. Biol., 6: 281-288, 1996. Methods of generating aptamers are known in the art and are described, for example, in U.S. Pat. Nos. 6,344,318, 6,331,398, 6,110,900, 5,817,785, 5,756,291, 5,696,249, 5,670,637, 5,637,461, 5,595,877, 5,527,894, 5,496,938, 5,475,096, 5,270,163, and in U.S. Patent Application Publication Nos. 20040241731, 20030198989, 20030157487, and 20020172962.

An aptamer of the present invention is capable of binding with specificity to a procollagen c-terminal propeptide expressed by a cell of interest. “Binding with specificity” means that non-procollagen c-terminal propeptides are either not specifically bound by the aptamer or are only poorly bound by the aptamer. In general, aptamers typically have binding constants in the picomolar range. Particularly useful in the methods of the present invention are aptamers having apparent dissociation constants of 1, 10, 15, 25, 50, 75, or 100 nM. Because many cells of interest express one or more procollagen c-terminal propeptides, in one embodiment, the present invention features a pharmaceutical composition that contains two or more aptamers, each of which recognizes a different procollagen c-terminal propeptide.

In one embodiment, a procollagen c-terminal propeptide (e.g. PICP) is the molecular target of the aptamer. Because aptamers can act as direct antagonists of the biological function of proteins, aptamers that target a procollagen c-terminal propeptide can be used to modulate angiogenesis, vasculogenesis, blood vessel stabilization or remodeling. The therapeutic benefit of such aptamers derives primarily from the biological antagonism caused by aptamer binding.

The present invention encompasses stabilized aptamers having modifications that protect against 3′ and 5′ exonucleases as well as endonucleases. Such modifications desirably maintain target affinity while increasing aptamer stability in vivo. In various embodiments, aptamers of the present invention include chemical substitutions at the ribose and/or phosphate and/or base positions of a given nucleobase sequence. For example, aptamers of the present invention include chemical modifications at the 2′ position of the ribose moiety, circularization of the aptamer, 3′ capping and “spiegelmer” technology. Such modifications are known in the art and are described herein. Aptamers having A and G nucleotides sequentially replaced with their 2′-OCH3 modified counterparts are particularly useful in the methods of the present invention. Such modifications are typically well tolerated in terms of retaining aptamer affinity and specificity. In various embodiments, aptamers include at least 10%, 25%, 50%, or 75% modified nucleotides. In other embodiments, as many as 80-90% of the aptatmer's nucleotides contain stabilizing substitutions. In other embodiments, 2′-OMe aptamers are synthesized. Such aptamers are desirable because they are inexpensive to synthesize and natural polymerases do not accept 2′-OMe nucleotide triphosphates as substrates so that 2′-OMe nucleotides cannot be recycled into host DNA. A fully 2′-O-methyl aptamer, named ARC245, was reported to be so stable that degradation could not be detected after 96 hours in plasma at 37.degree. C. or after autoclaving at 125.degree. C. Using methods, described herein, aptamers will be selected for reduced size and increased stability. In one embodiment, aptamers having 2′-F and 2′-OCH.sub.3 modifications are used to generate nuclease resistant aptamers. Other modifications that stabilize aptamers are known in the art and are described, for example, in U.S. Pat. No. 5,580,737; and in U.S. Patent Application Publication Nos. 20050037394, 20040253679, 20040197804, and 20040180360.

Using standard methods procollagen c-terminal propeptide-specific aptamers can be selected that bind virtually any procollagen c-terminal propeptide known in the art. Exemplary aptamers useful for targeting an angiogenic cell type include EYE0001, and those that target angiopoietin-2 (White et al., Proc Natl Acad Sci USA. 2003 Apr. 29; 100(9):5028-33 and pigpen (Blank et al., J. Biol. Chem. 2001 May 11; 276(19):16464-8).

### XVI. DELIVERY OF NUCLEOBASE OLIGOMERS

Naked inhibitory nucleic acid molecules, or analogs thereof; are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611; 5,753,613; 5,785,992; 6,120,798; 6,221,959; 6,346,613; and 6,353,055, each of which is hereby incorporated by reference).

### XVII. PHARMACEUTICAL COMPOSITIONS

The present invention contemplates pharmaceutical preparations comprising a procollagen c-terminal propeptide, a polynucleotide that encodes a procollagen c-terminal propeptide, an aptamer that binds a procollagen c-terminal propeptide, or a procollagen c-terminal propeptide inhibitory nucleic acid molecule (e.g., a polynucleotide that hybridizes to and interferes with the expression of a procollagen c-terminal propeptide polynucleotide), together with a pharmaceutically acceptable carrier. Polynucleotides of the present invention may be administered as part of a pharmaceutical composition. The compositions should be sterile and contain a therapeutically effective amount of the polypeptides or nucleic acid molecules in a unit of weight or volume suitable for administration to a subject.

These compositions ordinarily will be stored in unit or multi-dose containers, for example, sealed ampoules or vials, as an aqueous solution or as a lyophilized formulation for reconstitution. As an example of a lyophilized formulation, 10 mL vials are filled with 5 mL of sterile-filtered 1% (w/v) aqueous procollagen c-terminal propeptide polynucleotide solution, such as an aqueous solution of procollagen c-terminal propeptide polynucleotide or polypeptide, and the resulting mixture can then be lyophilized. The infusion solution can be prepared by reconstituting the lyophilized material using sterile Water-for-Injection (WFI).

The procollagen c-terminal propeptide polynucleotide, or polypeptide, or analogs may be combined, optionally, with a pharmaceutically acceptable excipient. The term “pharmaceutically-acceptable excipient” as used herein means one or more compatible solid or liquid filler, diluents or encapsulating substances that are suitable for administration into a human. The term “carrier” denotes an organic or inorganic ingredient, natural or synthetic, with which the active ingredient is combined to facilitate administration. The components of the pharmaceutical compositions also are capable of being co-mingled with the molecules of the present invention, and with each other, in a manner such that there is no interaction that would substantially impair the desired pharmaceutical efficacy.

The compositions can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject having a neoplastic disease or disorder, an effective amount is sufficient to stabilize, slow, or reduce the proliferation of the neoplasm. Generally, doses of active polynucleotide or polypeptide compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the procollagen c-terminal propeptide polynucleotide or polypeptide compositions of the present invention.

A variety of administration routes are available. The methods of the present invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes. A particular method of administration involves coating, embedding or derivatizing fibers, such as collagen fibers, protein polymers, etc. with therapeutic proteins. Other useful approaches are described in Otto, D. et al., J. Neurosci. Res. 22: 83-91 and in Otto, D. and Unsicker, K. J. Neurosci. 10: 1912-1921.

Nanoparticles are a colloidal carrier system that has been shown to improve the efficacy of the encapsulated drug by prolonging the serum half-life. Polyalkylcyanoacrylates (PACAs) nanoparticles are a polymer colloidal drug delivery system that is in clinical development, as described by Stella et al., J. Pharm. Sci., 2000. 89: p. 1452-1464; Brigger et al., Int. J. Pharm., 2001.214: p. 3742; Calvo et al., Pharm. Res., 2001. 18: p. 1157-1166; and Li et al., Biol. Pharm. Bull., 2001. 24: p. 662-665. Biodegradable poly (hydroxyl acids), such as the copolymers of poly(acetic acid) (PLA) and poly (lactic-o-glycolide) (PLGA) are being extensively used in biomedical applications and have received FDA approval for certain clinical applications. In addition, PEG-PLGA nanoparticles have many desirable carrier features including (i) that the agent to be encapsulated comprises a reasonably high weight fraction (loading) of the total carrier system; (ii) that the amount of agent used in the first step of the encapsulation process is incorporated into the final carrier (entrapment efficiency) at a reasonably high level; (iii) that the carrier have the ability to be freeze-dried and reconstituted in solution without aggregation; (iv) that the carrier be biodegradable; (v) that the carrier system be of small size; and (vi) that the carrier enhance the particles persistence.

Nanoparticles are synthesized using virtually any biodegradable shell known in the art. In one embodiment, a polymer, such as poly(lactic-acid) (PLA) or poly(lactic-co-glycolic acid) (PLGA) is used. Such polymers are biocompatible and biodegradable, and are subject to modifications that desirably increase the photochemical efficacy and circulation lifetime of the nanoparticle. In one embodiment, the polymer is modified with a terminal carboxylic acid group (COOH) that increases the negative charge of the particle and thus limits the interaction with negatively charge nucleic acid aptamers. Nanoparticles are also modified with polyethylene glycol (PEG), which also increases the half-life and stability of the particles in circulation. Alternatively, the COOH group is converted to an N-hydroxysuccinimide (NHS) ester for covalent conjugation to amine-modified aptamers.

Biocompatible polymers useful in the composition and methods of the present invention include, but are not limited to, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetage phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutylmethacrylate), poly(hexlmethacrylate), poly(isodecl methacrylate), poly(lauryl methacrylate), poly(phenyl methacrylate), poly(methyl acrylate), poly(isopropyl acrylate), poly(isobutyl acrylate), poly(octadecl acrylate) and combinations of any of these. In one embodiment, the nanoparticles of the present invention include PEG-PLGA polymers.

Compositions of the present invention may also be delivered topically. For topical delivery, the compositions are provided in any pharmaceutically acceptable excipient that is approved for ocular delivery. Preferably, the composition is delivered in drop form to the surface of the eye. For some application, the delivery of the composition relies on the diffusion of the compounds through the cornea to the interior of the eye.

Those of skill in the art will recognize that the best-treatment regimens for using compounds of the present invention to treat a disease characterized by, for example, pathological neovascularization can be straightforwardly determined. This is not a question of experimentation, but rather one of optimization, which is routinely conducted in the medical arts. In vivo studies in nude mice often provide a starting point from which to begin to optimize the dosage and delivery regimes. The frequency of injection will initially be once a week, as has been done in some mice studies. However, this frequency might be optimally adjusted from one day to every two weeks to monthly, depending upon the results obtained from the initial clinical trials and the needs of a particular patient.

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Where a composition of the present invention is used dosages of 1 mg, 2 mg, 3 mg, 5 mg, 7 mg, 10 mg, 15 mg, 20 mg, or 25 mg can be used per day. Of course, this dosage amount may be adjusted upward or downward, as is routinely done in such treatment protocols, depending on the results of the initial clinical trials and the needs of a particular patient. In various embodiments, compositions of the present invention are administered directly to a tissue or organ of interest by direct injection of a protein or inhibitory nucleic acid molecule described herein or by injection of a vector, such as a viral vector encoding a protein or inhibitory nucleic acid molecule of interest. In one approach, a therapeutic composition is administered in or near the target tissue.

### XVIII. SCREENING ASSAYS

As reported herein, the expression of a procollagen c-terminal propeptide (e.g., PICP) facilitates lumenized sprouting in the presence of proangiogenic growth factors. Accordingly, compounds that modulate the expression or activity of a procollagen c-terminal propeptide, variant, or fragment thereof are useful in the methods of the present invention for the treatment or prevention of a disease or disorder that requires modulation of angiogenesis, vasculogenesis, blood vessel stabilization or remodeling. Any number of methods are available for carrying out screening assays to identify such compounds. In one approach, candidate compounds are identified that specifically bind to and alter the activity of a polypeptide of the present invention (e.g., a procollagen c-terminal propeptide activity associated with angiogenesis, vasculogenesis, blood vessel stabilization or remodeling). Methods of assaying such biological activities are known in the art and are described herein. The efficacy of such a candidate compound is dependent upon its ability to interact with a procollagen c-terminal propeptide, variant, or fragment. Such an interaction can be readily assayed using any number of standard binding techniques and functional assays (e.g., those described in Ausubel et al., supra). For example, a candidate compound may be tested in vitro for interaction and binding with a polypeptide of the present invention and its ability to modulate angiogenesis, vasculogenesis, blood vessel stabilization or remodeling. Procollagen c-terminal propeptide's function in angiogenesis, vasculogenesis, blood vessel stabilization or remodeling can be assayed by detecting, for example, tube formation or extension in an endothelial cell where endogenous procollagen c-terminal propeptide expression or activity is perturbed or reduced. Standard methods for perturbing or reducing procollagen c-terminal propeptide expression include mutating or deleting an endogenous procollagen c-terminal propeptide sequence, interfering with procollagen c-terminal propeptide expression using RNAi, or microinjecting a procollagen c-terminal propeptide-expressing cell with an antibody or aptamer that binds procollagen c-terminal propeptide and interferes with its function. Alternatively, angiogenesis, vasculogenesis, blood vessel stabilization or remodeling can be assayed in vivo, for example, in a mouse model in which procollagen c-terminal propeptide has been knocked out by homologous recombination, or any other standard method.

Potential agonists and antagonists of a procollagen c-terminal propeptide include organic molecules, peptides, peptide mimetics, polypeptides, nucleic acid molecules (e.g., double-stranded RNAs, siRNAs, antisense polynucleotides, aptamers), and antibodies that bind to a nucleic acid sequence or polypeptide of the present invention and thereby inhibit or extinguish its activity. Potential antagonists also include small molecules that bind to the procollagen c-terminal propeptide thereby preventing binding to cellular molecules with which the procollagen c-terminal propeptide normally interacts (e.g., VEGF), such that the normal biological activity of the procollagen c-terminal propeptide is reduced or inhibited. Small molecules of the present invention preferably have a molecular weight below 2,000 daltons, more preferably between 300 and 1,000 daltons, and most preferably between 400 and 700 daltons. It is preferred that these small molecules are organic molecules.

In one particular example, a candidate compound that binds to a procollagen c-terminal propeptide, variant, or fragment thereof may be identified using a chromatography-based technique. For example, a recombinant polypeptide of the present invention may be purified by standard techniques from cells engineered to express the polypeptide (e.g., those described above) and may be immobilized on a column. A solution of candidate compounds is then passed through the column, and a compound specific for the procollagen c-terminal propeptide is identified on the basis of its ability to bind to the procollagen c-terminal propeptide and be immobilized on the column. To isolate the compound, the column is washed to remove non-specifically bound molecules, and the compound of interest is then released from the column and collected.

Similar methods may be used to isolate a compound bound to a polypeptide microarray. Compounds isolated by this method (or any other appropriate method) may, if desired, be further purified (e.g., by high performance liquid chromatography). In addition, these candidate compounds may be tested for their ability to alter the biological activity of a procollagen c-terminal propeptide.

Compounds that are identified as binding to a polypeptide of the present invention with an affinity constant less than or equal to about 10 mM are considered particularly useful in the present invention. Alternatively, any in vivo protein interaction detection system, for example, any two-hybrid assay may be utilized to identify compounds that interact with a procollagen c-terminal propeptide. Interacting compounds isolated by this method (or any other appropriate method) may, if desired, be further purified (e.g., by high performance liquid chromatography). Compounds isolated by any approach described herein may be used as therapeutics to treat a vascular disease in a human patient.

In addition, compounds that inhibit the expression of a procollagen c-terminal propeptide nucleic acid molecule whose expression is altered in a patient having a vascular disease or disorder are also useful in the methods of the present invention. Any number of methods are available for carrying out screening assays to identify new candidate compounds that alter the expression of a procollagen c-terminal propeptide nucleic acid molecule. In one working example, candidate compounds are added at varying concentrations to the culture medium of cultured cells expressing one of the nucleic acid sequences of the present invention. Gene expression is then measured, for example, by microarray analysis, Northern blot analysis (Ausubel et al., supra), or RT-PCR, using any appropriate fragment prepared from the nucleic acid molecule as a hybridization probe. The level of gene expression in the presence of the candidate compound is compared to the level measured in a control culture medium lacking the candidate molecule. A compound that promotes an alteration in the expression of a procollagen c-terminal propeptide gene, or a functional equivalent thereof, is considered useful in the present; such a molecule may be used, for example, as a therapeutic to treat a vascular disease or disorder in a human patient.

In another approach, the effect of candidate compounds is measured at the level of polypeptide production to identify those that promote an alteration in a procollagen c-terminal propeptide level. The level of procollagen c-terminal propeptide can be assayed using any standard method. Standard immunological techniques include Western blotting or immunoprecipitation with an antibody specific for a procollagen c-terminal propeptide. For example, immunoassays may be used to detect or monitor the expression of at least one of the polypeptides of the present invention in an organism. Polyclonal or monoclonal antibodies (produced as described above) that are capable of binding to such a polypeptide may be used in any standard immunoassay format (e.g., ELISA, Western blot, or RIA assay) to measure the level of the polypeptide. In some embodiments, a compound that promotes a decrease in the expression or biological activity of the polypeptide is considered particularly useful. Again, such a molecule may be used, for example, as a therapeutic to delay, ameliorate, or treat a vascular disease in a human patient.

In another embodiment, a nucleic acid described herein is expressed as a transcriptional or translational fusion with a detectable reporter, and expressed in an isolated cell (e.g., mammalian or insect cell) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that alters the expression of the detectable reporter is a compound that is useful for the treatment of vascular disease. In one embodiment, the compound decreases the expression of the reporter.

Each of the DNA sequences referenced herein may also be used in the discovery and development of a therapeutic compound for the treatment of vascular disease. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

The present invention also includes novel compounds identified by the above-described screening assays. Optionally, such compounds are characterized in one or more appropriate animal models to determine the efficacy of the compound for the treatment of a vascular disease. Desirably, characterization in an animal model can also be used to determine the toxicity, side effects, or mechanism of action of treatment with such a compound. Furthermore, novel compounds identified in any of the above-described screening assays may be used for the treatment of a vascular disease in a subject. Such compounds are useful alone or in combination with other conventional therapies known in the art.

### XIX. TEST COMPOUNDS AND EXTRACTS

In general, compounds capable of inhibiting the growth or proliferation of a vascular disease by altering the expression or biological activity of a procollagen c-terminal propeptide, variant, or fragment thereof are identified from large libraries of either natural product or synthetic (or semi-synthetic) extracts or chemical libraries according to methods known in the art. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmalMar, U.S. (Cambridge, Mass.).

In one embodiment, test compounds of the present invention are present in any combinatorial library known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al., J. Med. Chem. 37:2678-85, 1994); spatially addressable parallel solid phase or solution phase libraries; synthetic library methods requiring deconvolution; the “one-bead one-compound” library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer. DrugDes. 12:145, 1997).

Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-neoplastic activity should be employed whenever possible.

Those skilled in the field of drug discovery and development will understand that the precise source of a compound or test extract is not critical to the screening procedure(s) of the present invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

When a crude extract is found to alter the biological activity of a procollagen c-terminal propeptide, variant, or fragment thereof, further fractionation of the positive lead extract is necessary to isolate chemical constituents responsible for the observed effect. Thus, the goal of the extraction, fractionation, and purification process is the careful characterization and identification of a chemical entity within the crude extract having anti-neoplastic activity. Methods of fractionation and purification of such heterogeneous extracts are known in the art. If desired, compounds shown to be useful agents for the treatment of vascular disease are chemically modified according to methods known in the art.

### XX. KITS OR PHARMACEUTICAL SYSTEMS

The present compositions may be assembled into kits or pharmaceutical systems for use in ameliorating vascular disease. Kits or pharmaceutical systems according to this aspect of the present invention comprise a carrier means, such as a box, carton, tube or the like, having in close confinement therein one or more container means, such as vials, tubes, ampules, bottles and the like. The kits or pharmaceutical systems of the present invention may also comprise associated instructions for using the agents of the present invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the present invention, and, as such, may be considered in making and practicing the present invention. Particularly useful techniques for particular embodiments will be discussed in the sections that follow.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Procollagen (I) C-Terminal Propeptide (PICP) Expression and Isolation

To isolate the native heterotrimeric c-terminal propeptide conditioned serum-free media (Lonza) from confluent lung fibroblasts (Lonza) were collected for 24-48 h. Media was concentrated on Millipore 100 kda molecular weight filters (Millipore Corp.) tubes. Concentrated media was then dialyzed at 4° C. against 50 mM Tris-HCl, pH7.5, and loaded on a 2.5-ml column of heparin-Sepharose and eluted stepwise or with a linear gradient of NaCl (0-1 M) in Tris-HCl 50 mM, pH 7.5, at 4° C. The fractions active in the bioassay were pooled and dialyzed against 50 mmol/L sodium phosphate buffer, pH7.5, containing proteinase inhibitors such as PMSF (1 mM) and 2.0 mol/L (NH₄)₂SO₄ and loaded on a 2 ml thiophilic agarose column (Clontech). Protein was then eluted at a gradient of 1.5-0.0 mol/L (NH₄)₂SO₄ in 100 mL and fractions were collected. Samples were run on a reducing SDS-PAGE and stained with Gelcode Blue to identify target protein bands around 30 kda and assess purity. If required further purification using gel-filtration with a Sephacryl S-300 column can be performed.

### Example 2

### Expression of Recombinant Homotrimeric PICP

To express recombinant homotrimeric PICP, the gene (Open Biosystems) product encoding the c-terminal propeptide (amino acid 1219-1464 of the col1a1 protein) was cloned into a lentiviral expression vector containing puromycin as a selection marker (Clontech). Different tags such as 6×His, FLAG peptide or IgG2a were added to the terminal end of the coding sequence. The signal peptide sequence of the col1a1 gene (amino acid 1-22) or a recently described signal peptide of the gaussia luciferase (ATGGGAGTGAAAGTTCTTTTTGCCCTTATTTGTATTGCTGTGGC CGAGGCC) (SEQ ID NO:13) was fused to the n-terminal position of the coding sequence for the PICP fragment of the collagen 1 molecule. The PCR product was either digested with restriction enzymes to allow for the directional cloning into the multiple cutting site of the vector or integrated into the cut vector using the Clontech In-Fusion PCR cloning system following the manufacturer instructions. Lentiviral expression vectors were mixed with a packaging and envelope vector (Trono lab, Addgene) and used to transfect the HEK293T/17 cells (ATCC) with standard calcium precipitation to generate lentiviral particles. Lentiviral particles were collected and used to transduce HEK293S cells (Invitrogen). Cells were cultured in serum free HEK293 media (Sigma) and kept under suspension conditions. Stable cell lines were produced by puromycin selection (10 microgram/ml). Conditioned media was collected and the target protein purified using the respective affinity chromatography methods (Ni-column, M2 Agarose and IgG2a) as described by the manufacturer.

### Example 3

### Inhibition of PICP Activity

As a proof of principle that targeting the PICP pathway can inhibit fibroblast supported blood vessel formation, an inactive mutant of PICP was developed. PICP has a highly conserved N-glycosylation site at amino acid 1365 of the col1a1 protein. The importance of this site for inducing proangiogenic activity has been unknown. The expression vector containing the His-tagged PICP coding sequence was targeted for site directed mutagenesis. The asparagine at the 1365 position was mutated to an alanine (Forward Primer: ATGTCCACCGAGGCCTCCCAGGCCATCACCTACCACTGCA AGAAC (SEQ ID NO:14), Reverse Primer: GTTCTTGCAGTGGTAGGTGATGGCCTGGG AGGCCTCGGT GGACAT (SEQ ID NO:15)). The mutation was confirmed using PCR. The production of the lentiviral particles and generation of stable cell lines was performed as above. The mutated PICP (PICPmut) was added to wells containing fibroblast-conditioned media and sprouting was significantly suppressed. Controls from the conditioned media of HEK293 cells had no suppressive effect.

### Example 4

### In Vitro Angiogenesis Assay

The assay was used as previously described. See Nakatsu et al., 66(2) MICROVASC. RES. 102-12 (2003). Human umbilical vein endothelial cells (HUVEC) were seeded onto dextran beads and embedded into a fibrin matrix. In a variation the assay was performed in 96 well plates containing 30 ul fibrin/bead matric and 70 ul of EGM2 media. Recombinant VEGF (500 ng/ml) with either purified or enriched fractions of PICP or concentrated (10×) conditioned media derived from lung fibroblasts (positive control) was added the endothelial cell media (EGM-2, Cambrex). Vascular sprouts were typically grown for 7-10 days. Growth inhibitory experiments were performed by adding 30 microliters enriched (10× Concentrated) conditioned media from either wildtype or PICPmut expressing HEK293S cells.

1. A method for modulating a blood vessel in a subject in need thereof comprising contacting a cell of the subject with a procollagen carboxy-terminal propeptide, a biologically active fragment or mimetic thereof, thereby modulating the blood vessel.

2. The method of claim 1, further comprising contacting a cell of the subject with one or more endothelial growth factors.

3. The method of claim 2, wherein the one or more endothelial growth factors is vascular endothelial growth factor.

4. The method of claim 1, wherein the method increases or decreases blood vessel formation relative to an untreated control tissue or organ.

5. The method of claim 1, wherein the method stabilizes or remodels a blood vessel in a tissue or organ relative to an untreated control tissue or organ.

6. The method of claim 1, wherein the procollagen c-terminal propeptide is selected from the group consisting of collagen I, collagen II, collagen III, collagen V, collagen XI, collagen XXIV, and collagen XXVII.

7. The method of claim 1, wherein the procollagen c-terminal propeptide is collagen I.

8. A method for decreasing angiogenesis in a subject in need thereof comprising contacting a cell of the subject with an agent that inhibits the expression or biological activity of a procollagen carboxy-terminal propeptide.

9. The method of claim 8, wherein the subject has a disease, disorder, or tissue damage and the contacting step ameliorates the disease, disorder, or tissue damage.

10. A method of treating pathological neovascularization in a subject comprising administering to the subject an agent that decreases angiogenesis in the subject, thereby treating pathological neovascularization in the subject.

11. The method of claim 8, wherein the method decreases angiogenesis in a tissue or organ of the subject by at least 5% compared to an untreated control tissue or organ.

12. The method of claim 11, wherein the tissue is a neoplastic tissue.

13. The method of claim 8, wherein the cell, tissue or organ is selected from the group consisting of brain, nervous tissue, eye, ocular tissue, heart, cardiac tissue, and skeletal muscle tissue bladder, bone, brain, breast, cartilage, nervous tissue, esophagus, fallopian tube, heart, pancreas, intestines, gallbladder, kidney, liver, lung, ovaries, prostate, skeletal muscle, skin, spinal cord, spleen, stomach, testes, thymus, thyroid, trachea, urogenital tract, ureter, urethra, and uterus.

14. The method of claim 8, wherein the agent is an antibody or an aptamer that binds a procollagen c-terminal propeptide.

15. The method of claim 8, wherein the agent is an inhibitory nucleic acid molecule that decreases the expression of a procollagen c-terminal propeptide.

16. The method of claim 15, wherein the inhibitory nucleic acid molecule is an antisense oligonucleotide, a short interfering RNA (siRNA), or a short hairpin RNA (shRNA).

17. The method of claim 1, wherein the subject is a human.

18. A method for increasing blood vessel formation in a tissue or organ comprising contacting a cell of the tissue or organ with a procollagen c-terminal propeptide, biologically active fragment or mimetic thereof, thereby increasing blood vessel formation in the tissue or organ.

19. A method for stabilizing a blood vessel in a tissue or organ comprising contacting a cell of the tissue or organ with a procollagen c-terminal propeptide, biologically active fragment, or mimetic thereof, thereby stabilizing a blood vessel in the subject.

20. A method for increasing blood vessel formation or stabilizing or remodeling a blood vessel in a tissue or organ comprising contacting a cell of the tissue or organ with a nucleic acid molecule encoding a procollagen c-terminal propeptide, biologically active fragment, or mimetic thereof, thereby increasing blood vessel formation or stabilizing or remodeling a blood vessel in a tissue or organ.

21. The method of claim 18, wherein the contacting increases blood vessel formation or stabilizes a blood vessel in a tissue or organ of a subject.

22. The method of claim 21, wherein the tissue or organ is selected from the group consisting of bladder, bone, breast, cartilage, esophagus, fallopian tube, pancreas, intestines, gallbladder, kidney, liver, lung, ovaries, prostate, skin, spinal cord, spleen, stomach, testes, thymus, thyroid, trachea, urogenital tract, ureter, urethra, uterus, brain, nervous tissue, eye, ocular tissue, heart, cardiac tissue, and skeletal muscle tissue.

23. The method of claim 1, wherein the contacting occurs in vitro or in vivo.

24. The method of claim 1, wherein the cell is a human cell.

25. The method of claim 1, wherein the cell is an endothelial cell, pericyte, muscle cell, neuron or a glial cell.

26. The method of claim 1, wherein the cell is present in a subject that has a disease, disorder, or tissue damage and the contacting ameliorates the disease, disorder, or tissue damage.

27. An inhibitory nucleic acid molecule that specifically binds at least a fragment of a nucleic acid molecule encoding a procollagen c-terminal propeptide and decreases the expression of the procollagen c-terminal propeptide.

28. The inhibitory nucleic acid molecule of claim 27, wherein the inhibitory nucleic acid molecule is an siRNA, an antisense oligonucleotide, an shRNA, or a ribozyme.

29. An aptamer that specifically binds at least a fragment of a procollagen c-terminal propeptide and decreases a biological activity of the procollagen c-terminal propeptide.

30. A vector comprising a nucleic acid molecule encoding a procollagen c-terminal propeptide, biologically active fragment or mimetic thereof, or encoding the inhibitory nucleic acid molecule of claim 27, wherein the nucleic acid molecule is positioned for expression.

31. The vector of claim 30, wherein the nucleic acid molecule is operably linked to a promoter suitable for expression in a mammalian cell.

32. A host cell comprising the nucleic acid molecule of claim 27.

33. The host cell of claim 32, wherein the cell is a human cell.

34. The host cell of claim 32, wherein the cell is in vitro or in vivo.

35. A pharmaceutical composition for modulating a blood vessel in a subject comprising an effective amount of a procollagen c-terminal propeptide, biologically active fragment or mimetic thereof in a pharmaceutically acceptable excipient.

36. A pharmaceutical composition for modulating a blood vessel in a subject comprising an effective amount of an inhibitory nucleic acid molecule of claim 27 that reduces the expression of a procollagen c-terminal propeptide in a pharmaceutically acceptable excipient.

37. A pharmaceutical composition for modulating a blood vessel in a subject comprising an effective amount of an aptamer that specifically binds a procollagen c-terminal propeptide or biologically active fragment thereof in a pharmaceutically acceptable excipient.

38. A pharmaceutical composition for modulating a blood vessel in a subject comprising an effective amount of an antibody that specifically binds a procollagen c-terminal propeptide or biologically active fragment thereof in a pharmaceutically acceptable excipient.

39. A pharmaceutical composition comprising an effective amount of a vector comprising a nucleic acid molecule encoding a procollagen c-terminal propeptide or biologically active fragment in a pharmaceutically acceptable excipient, wherein expression of the propeptide in a cell is capable of modulating a blood vessel.

40-63. (canceled)

64. A method for prevascularizing a tissue graft comprising contacting a cell of the tissue with a procollagen carboxy-terminal propeptide, a biologically active fragment or mimetic thereof, thereby prevascularizing the tissue graft.

65. The method of claim 64, further comprising contacting a cell of the subject with one or more endothelial growth factors.

66. The method of claim 65, wherein the one or more endothelial growth factors is vascular endothelial growth factor.

67-68. (canceled)


SEQUENCE LISTING (15 sequences)

SEQ ID NO: 1 - protein, 1464 aa, Homo sapiens
  MISC_FEATURE (1)..(1464): Human collagen alpha-1(I) chain
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPTGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 246 aa, Homo sapiens
  PROPEP (1)..(246): Procollagen (I) c-terminal propeptide (PICP)
DDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 3 - protein, 1466 aa, Homo sapiens
  MISC_FEATURE (1)..(1466): Human collagen alpha-1(III) chain
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 4 - protein, 245 aa, Homo sapiens
  PROPEP (1)..(245): Procollagen (III) c-terminal propeptide (PIIICP)
DEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 5 - protein, 1838 aa, Homo sapiens
  MISC_FEATURE (1)..(1838): Human collagen alpha-1(V) chain
MDVHTRWKARSALRPGAPLLPPLLLLLLWAPPPSRAAQPADLLKVLDFHNLPDGITKTTGFCATRRSSKGPDVAYRVTKDAQLSAPTKQLYPASAFPEDFSILTTVKAKKGSQAFLVSIYNEQGIQQIGLELGRSPVFLYEDHTGKPGPEDYPLFRGINLSDGKWHRIALSVHKKNVTLILDCKKKTTKFLDRSDHPMIDINGIIVFGTRILDEEVFEGDIQQLLFVSDHRAAYDYCEHYSPDCDTAVPDTPQSQDPNPDEYYTEGDGEGETYYYEYPYYEDPEDLGKEPTPSKKPVEAAKETTEVPEELTPTPTEAAPMPETSEGAGKEEDVGIGDYDYVPSEDYYTPSPYDDLTYGEGEENPDQPTDPGAGAEIPTSTADTSNSSNPAPPPGEGADDLEGEFTEETIRNLDENYYDPYYDPTSSPSEIGPGMPANQDTIYEGIGGPRGEKGQKGEPAIIEPGMLIEGPPGPEGPAGLPGPPGTMGPTGQVGDPGERGPPGRPGLPGADGLPGPPGTMLMLPFRFGGGGDAGSKGPMVSAQESQAQAILQQARLALRGPAGPMGLTGRPGPVGPPGSGGLKGEPGDVGPQGPRGVQGPPGPAGKPGRRGRAGSDGARGMPGQTGPKGDRGFDGLAGLPGEKGHRGDPGPSGPPGPPGDDGERGDDGEVGPRGLPGEPGPRGLLGPKGPPGPPGPPGVTGMDGQPGPKGNVGPQGEPGPPGQQGNPGAQGLPGPQGAIGPPGEKGPLGKPGLPGMPGADGPPGHPGKEGPPGEKGGQGPPGPQGPIGYPGPRGVKGADGIRGLKGTKGEKGEDGFPGFKGDMGIKGDRGEIGPPGPRGEDGPEGPKGRGGPNGDPGPLGPPGEKGKLGVPGLPGYPGRQGPKGSIGFPGFPGANGEKGGRGTPGKPGPRGQRGPTGPRGERGPRGITGKPGPKGNSGGDGPAGPPGERGPNGPQGPTGFPGPKGPPGPPGKDGLPGHPGQRGETGFQGKTGPPGPPGVVGPQGPTGETGPMGERGHPGPPGPPGEQGLPGLAGKEGTKGDPGPAGLPGKDGPPGLRGFPGDRGLPGPVGALGLKGNEGPPGPPGPAGSPGERGPAGAAGPIGIPGRPGPQGPPGPAGEKGAPGEKGPQGPAGRDGLQGPVGLPGPAGPVGPPGEDGDKGEIGEPGQKGSKGDKGEQGPPGPTGPQGPIGQPGPSGADGEPGPRGQQGLFGQKGDEGPRGFPGPPGPVGLQGLPGPPGEKGETGDVGQMGPPGPPGPRGPSGAPGADGPQGPPGGIGNPGAVGEKGEPGEAGEPGLPGEGGPPGPKGERGEKGESGPSGAAGPPGPKGPPGDDGPKGSPGPVGFPGDPGPPGEPGPAGQDGPPGDKGDDGEPGQTGSPGPTGEPGPSGPPGKRGPPGPAGPEGRQGEKGAKGEAGLEGPPGKTGPIGPQGAPGKPGPDGLRGIPGPVGEQGLPGSPGPDGPPGPMGPPGLPGLKGDSGPKGEKGHPGLIGLIGPPGEQGEKGDRGLPGPQGSSGPKGEQGITGPSGPIGPPGPPGLPGPPGPKGAKGSSGPTGPKGEAGHPGPPGPPGPPGEVIQPLPIQASRTRRNIDASQLLDDGNGENYVDYADGMEEIFGSLNSLKLEIEQMKRPLGTQQNPARTCKDLQLCHPDFPDGEYWVDPNQGCSRDSFKVYCNFTAGGSTCVFPDKKSEGARITSWPKENPGSWFSEFKRGKLLSYVDAEGNPVGVVQMTFLRLLSASAHQNVTYHCYQSVAWQDAATGSYDKALRFLGSNDEEMSYDNNPYIRALVDGCATKKGYQKTVLEIDTPKVEQVPIVDIMFNDFGEASQKFGFEVGPACFMG

SEQ ID NO: 6 - protein, 233 aa, Homo sapiens
  PROPEP (1)..(233): Procollagen (V) c-terminal propeptide (PVCP)
DGMEEIFGSLNSLKLEIEQMKRPLGTQQNPARTCKDLQLCHPDFPDGEYWVDPNQGCSRDSFKVYCNFTAGGSTCVFPDKKSEGARITSWPKENPGSWFSEFKRGKLLSYVDAEGNPVGVVQMTFLRLLSASAHQNVTYHCYQSVAWQDAATGSYDKALRFLGSNDEEMSYDNNPYIRALVDGCATKKGYQKTVLEIDTPKVEQVPIVDIMFNDFGEASQKFGFEVGPACFMG

SEQ ID NO: 7 - protein, 1806 aa, Homo sapiens
  MISC_FEATURE (1)..(1806): Human collagen alpha-1(XI) chain
MEPWSSRWKTKRWLWDFTVTTLALTFLFQAREVRGAAPVDVLKALDFHNSPEGISKTTGFCTNRKNSKGSDTAYRVSKQAQLSAPTKQLFPGGTFPEDFSILFTVKPKKGIQSFLLSIYNEHGIQQIGVEVGRSPVFLFEDHTGKPAPEDYPLFRTVNIADGKWHRVAISVEKKTVTMIVDCKKKTTKPLDRSERAIVDTNGITVFGTRILDEEVFEGDIQQFLITGDPKAAYDYCEHYSPDCDSSAPKAAQAQEPQIDEYAPEDIIEYDYEYGEAEYKEAESVTEGPTVTEETIAQTEANIVDDFQEYNYGTMESYQTEAPRHVSGTNEPNPVEEIFTEEYLTGEDYDSQRKNSEDTLYENKEIDGRDSDLLVDGDLGEYDFYEYKEYEDKPTSPPNEEFGPGVPAETDITETSINGHGAYGEKGQKGEPAVVEPGMLVEGPPGPAGPAGIMGPPGLQGPTGPPGDPGDRGPPGRPGLPGADGLPGPPGTMLMLPFRYGGDGSKGPTISAQEAQAQAILQQARIALRGPPGPMGLTGRPGPVGGPGSSGAKGESGDPGPQGPRGVQGPPGPTGKPGKRGRPGADGGRGMPGEPGAKGDRGFDGLPGLPGDKGHRGERGPQGPPGPPGDDGMRGEDGEIGPRGLPGEAGPRGLLGPRGTPGAPGQPGMAGVDGPPGPKGNMGPQGEPGPPGQQGNPGPQGLPGPQGPIGPPGEKGPQGKPGLAGLPGADGPPGHPGKEGQSGEKGALGPPGPQGPIGYPGPRGVKGADGVRGLKGSKGEKGEDGFPGFKGDMGLKGDRGEVGQIGPRGEDGPEGPKGRAGPTGDPGPSGQAGEKGKLGVPGLPGYPGRQGPKGSTGFPGFPGANGEKGARGVAGKPGPRGQRGPTGPRGSRGARGPTGKPGPKGTSGGDGPPGPPGERGPQGPQGPVGFPGPKGPPGPPGKDGLPGHPGQRGETGFQGKTGPPGPGGVVGPQGPTGETGPIGERGHPGPPGPPGEQGLPGAAGKEGAKGDPGPQGISGKDGPAGLRGFPGERGLPGAQGAPGLKGGEGPQGPPGPVGSPGERGSAGTAGPIGLPGRPGPQGPPGPAGEKGAPGEKGPQGPAGRDGVQGPVGLPGPAGPAGSPGEDGDKGEIGEPGQKGSKGDKGENGPPGPPGLQGPVGAPGIAGGDGEPGPRGQQGMFGQKGDEGARGFPGPPGPIGLQGLPGPPGEKGENGDVGPMGPPGPPGPRGPQGPNGADGPQGPPGSVGSVGGVGEKGEPGEAGNPGPPGEAGVGGPKGERGEKGEAGPPGAAGPPGAKGPPGDDGPKGNPGPVGFPGDPGPPGEPGPAGQDGVGGDKGEDGDPGQPGPPGPSGEAGPPGPPGKRGPPGAAGAEGRQGEKGAKGEAGAEGPPGKTGPVGPQGPAGKPGPEGLRGIPGPVGEQGLPGAAGQDGPPGPMGPPGLPGLKGDPGSKGEKGHPGLIGLIGPPGEQGEKGDRGLPGTQGSPGAKGDGGIPGPAGPLGPPGPPGLPGPQGPKGNKGSTGPAGQKGDSGLPGPPGSPGPPGEVIQPLPILSSKKTRRHTEGMQADADDNILDYSDGMEEIFGSLNSLKQDIEHMKFPMGTQTNPARTCKDLQLSHPDFPDGEYWIDPNQGCSGDSFKVYCNFTSGGETCIYPDKKSEGVRISSWPKEKPGSWFSEFKRGKLLSYLDVEGNSINMVQMTFLKLLTASARQNFTYHCHQSAAWYDVSSGSYDKALRFLGSNDEEMSYDNNPFIKTLYDGCASRKGYEKTVIEINTPKIDQVPIVDVMINDFGDQNQKFGFEVGPVCFLG

SEQ ID NO: 8 - protein, 243 aa, Homo sapiens
  MISC_FEATURE (1)..(243): Procollagen (XI) c-terminal propeptide (PXICP)
DADDNILDYSDGMEEIFGSLNSLKQDIEHMKFPMGTQTNPARTCKDLQLSHPDFPDGEYWIDPNQGCSGDSFKVYCNFTSGGETCIYPDKKSEGVRISSWPKEKPGSWFSEFKRGKLLSYLDVEGNSINMVQMTFLKLLTASARQNFTYHCHQSAAWYDVSSGSYDKALRFLGSNDEEMSYDNNPFIKTLYDGCASRKGYEKTVIEINTPKIDQVPIVDVMINDFGDQNQKFGFEVGPVCFLG

SEQ ID NO: 9 - protein, 1860 aa, Homo sapiens
  MISC_FEATURE: Collagen alpha-1(XXVII) chain
MGAGSARGARGTAAAAAARGGGFLFSWILVSFACHLASTQGAPEDVDILQRLGLSWTKAGSPAPPGVIPFQSGFIFTQRARLQAPTGTVIPAALGTELALVLSLCSHRVNHAFLFAVRSQKRKLQLGLQFLPGKTVVHLGSRRSVAFDLDMHDGRWHHLALELRGRTVTLVTACGQRRVPVLLPFHRDPALDPGGSFLFGKMNPHAVQFEGALCQFSIYPVTQVAHNYCTHLRKQCGQADTYQSPLGPLFSQDSGRPFTFQSDLALLGLENLTTATPALGSLPAGRGPRGTVAPATPTKPQRTSPTNPHQHMAVGGPAQTPLLPAKLSASNALDPMLPASVGGSTRTPRPAAAQPSQKITATKIPKSLPTKPSAPSTSIVPIKSPHPTQKTAPSSFTKSALPTQKQVPPTSRPVPARVSRPAEKPIQRNPGMPRPPPPSTRPLPPTTSSSKKPIPTLARTEAKITSHASKPASARTSTHKPPPFTALSSSPAPTPGSTRSTRPPATMVPPTSGTSTPRTAPAVPTPGSAPTGSKKPIGSEASKKAGPKSSPRKPVPLRPGKAARDVPLSDLTTRPSPRQPQPSQQTTPALVLAPAQFLSSSPRPTSSGYSIFHLAGSTPFPLLMGPPGPKGDCGLPGPPGLPGLPGIPGARGPRGPPGPYGNPGLPGPPGAKGQKGDPGLSPGKAHDGAKGDMGLPGLSGNPGPPGRKGHKGYPGPAGHPGEQGQPGPEGSPGAKGYPGRQGLPGPVGDPGPKGSRGYIGLPGLFGLPGSDGERGLPGVPGKRGKMGMPGFPGVFGERGPPGLDGNPGELGLPGPPGVPGLIGDLGVLGPIGYPGPKGMKGLMGSVGEPGLKGDKGEQGVPGVSGDPGFQGDKGSQGLPGFPGARGKPGPLGKVGDKGSIGFPGPPGPEGFPGDIGPPGDNGPEGMKGKPGARGLPGPRGQLGPEGDEGPMGPPGAPGLEGQPGRKGFPGRPGLDGVKGEPGDPGRPGPVGEQGFMGFIGLVGEPGIVGEKGDRGMMGPPGVPGPKGSMGHPGMPGGMGTPGEPGPQGPPGSRGPPGMRGAKGRRGPRGPDGPAGEQGSRGLKGPPGPQGRPGRPGQQGVAGERGHLGSRGFPGIPGPSGPPGTKGLPGEPGPQGPQGPIGPPGEMGPKGPPGAVGEPGLPGEAGMKGDLGPLGTPGEQGLIGQRGEPGLEGDSGPMGPDGLKGDRGDPGPDGEHGEKGQEGLMGEDGPPGPPGVTGVRGPEGKSGKQGEKGRTGAKGAKGYQGQLGEMGVPGDPGPPGTPGPKGSRGSLGPTGAPGRMGAQGEPGLAGYDGHKGIVGPLGPPGPKGEKGEQGEDGKAEGPPGPPGDRGPVGDRGDRGEPGDPGYPGQEGVQGLRGKPGQQGQPGHPGPRGWPGPKGSKGAEGPKGKQGKAGAPGRRGVQGLQGLPGPRGVVGRQGLEGIAGPDGLPGRDGQAGQQGEQGDDGDPGPMGPAGKRGNPGVAGLPGAQGPPGFKGESGLPGQLGPPGKRGTEGRTGLPGNQGEPGSKGQPGDSGEMGFPGMAGLFGPKGPPGDIGFKGIQGPRGPPGLMGKEGIVGPLGILGPSGLPGPKGDKGSRGDWGLQGPRGPPGPRGRPGPPGPPGGPIQLQQDDLGAAFQTWMDTSGALRPESYSYPDRLVLDQGGEIFKTLHYLSNLIQSIKTPLGTKENPARVCRDLMDCEQKMVDGTYWVDPNLGCSSDTIEVSCNFTHGGQTCLKPITASKVEFAISRVQMNFLHLLSSEVTQHITIHCLNMTVWQEGTGQTPAKQAVRFRAWNGQIFEAGGQFRPEVSMDGCKVQDGRWHQTLFTFRTQDPQQLPIISVDNLPPASSGKQYRLEVGPACFL

SEQ ID NO: 10 - protein, 239 aa, Homo sapiens
  MISC_FEATURE (1)..(239): Procollagen (XXVII) c-terminal propeptide (PXXVIICP)
IQLQQDDLGAAFQTWMDTSGALRPESYSYPDRLVLDQGGEIFKTLHYLSNLIQSIKTPLGTKENPARVCRDLMDCEQKMVDGTYWVDPNLGCSSDTIEVSCNFTHGGQTCLKPITASKVEFAISRVQMNFLHLLSSEVTQHITIHCLNMTVWQEGTGQTPAKQAVRFRAWNGQIFEAGGQFRPEVSMDGCKVQDGRWHQTLFTFRTQDPQQLPIISVDNLPPASSGKQYRLEVGPACFL

SEQ ID NO: 11 - protein, 1487 aa, Homo sapiens
  MISC_FEATURE (1)..(1487): Human collagen alpha-1(II) chain
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 12 - protein, 1714 aa, Homo sapiens
  MISC_FEATURE (1)..(1714): Human collagen alpha-1(XXIV) chain
MHLRAHRTRRGKVSPTAKTKSLLHFIVLCVAGVVVHAQEQGIDILHQLGLGGKDVRHSSPATAVPSASTPLPQGVHLTESGVIFKNDAYIETPFVKILPVNLGQPFTILTGLQSHRVNNAFLFSIRNKNRLQLGVQLLPKKLVVHIRGKQPAVFNYSVHDEQWHSFAITIRNQSVSMFVECGKKYFSTETIPEVQTFDSNSVFTLGSMNNNSIHFEGIVCQLDIIPSAEASADYCRYVKQQCRQADKYQPETSIPCTTLIPTKIPEHSPPPKLFAEKVLSEDTFTEGKSIPNIIKNDSETVYKRQEHQISRSQLSSLQSGNVSAVDLTNHGIQAKEMITEEDTQTNFSLSVTTHRISEAKMNTKEKFSSLLNMSDNITQHDDRVTGLSLFKKMPSILPQIKQDTITNLKKAITANLHTNELMEMQPILNTSLHRVTNEPSVDNHLDLRKEGEFYPDATYPIENSYETELYDYYYYEDLNTMLEMEYLRGPKGDTGPPGPPGPAGIPGPSGKRGPRGIPGPHGNPGLPGLPGPKGPKGDPGFSPGQPVPGEKGDQGLSGLMGPPGMQGDKGLKGHPGLPGLPGEQGIPGFAGNIGSPGYPGRQGLAGPEGNPGPKGAQGFIGSPGEAGQLGPEGERGIPGIRGKKGFKGRQGFPGDFGDRGPAGLDGSPGLVGGTGPPGFPGLRGSVGPVGPIGPAGIPGPMGLSGNKGLPGIKGDKGEQGTAGELGEPGYPGDKGAVGLPGPPGMRGKSGPSGQTGDPGLQGPSGPPGPEGFPGDIGIPGQNGPEGPKGLLGNRGPPGPPGLKGTQGEEGPIGAFGELGPRGKPGQKGYAGEPGPEGLKGEVGDQGNIGKIGETGPVGLPGEVGMTGSIGEKGERGSPGPLGPQGEKGVMGYPGPPGVPGPIGPLGLPGHVGARGPPGSQGPKGQRGSRGPDGLLGEQGIQGAKGEKGDQGKRGPHGLIGKTGNPGERGFQGKPGLQGLPGSTGDRGLPGEPGLRGLQGDVGPPGEMGMEGPPGTEGESGLQGEPGAKGDVGTAGSVGGTGEPGLRGEPGAPGEEGLQGKDGLKGVPGGRGLPGEDGEKGEMGLPGIIGPLGRSGQTGLPGPEGIVGIPGQRGRPGKKGDKGQIGPTGEVGSRGPPGKIGKSGPKGARGTRGAVGHLGLMGPDGEPGIPGYRGHQGQPGPSGLPGPKGEKGYPGEDSTVLGPPGPRGEPGPVGDQGERGEPGAEGYKGHVGVPGLRGATGQQGPPGEPGDQGEQGLKGERGSEGNKGKKGAPGPSGKPGIPGLQGLLGPKGIQGYHGADGISGNPGKIGPPGKQGLPGIRGGPGRTGLAGAPGPPGVKGSSGLPGSPGIQGPKGEQGLPGQPGIQGKRGHRGAQGDQGPCGDPGLKGQPGEYGVQGLTGFQGFPGPKGPEGDAGIVGISGPKGPIGHRGNTGPLGREGIIGPTGRTGPRGEKGFRGETGPQGPRGQPGPPGPPGAPGPRKQMDINAAIQALIESNTALQMESYQNTEVTLIDHSEEIFKTLNYLSNLLHSIKNPLGTRDNPARICKDLLNCEQKVSDGKYWIDPNLGCPSDAIEVFCNFSAGGQTCLPPVSVTKLEFGVGKVQMNFLHLLSSEATHIITIHCLNTPRWTSTQTSGPGLPIGFKGWNGQIFKVNTLLEPKVLSDDCKIQDGSWHKATFLFHTQEPNQLPVIEVQKLPHLKTERKYYIDSSSVCFL

SEQ ID NO: 13 - DNA, 51 bp, Gaussia princeps
  misc_feature (1)..(51): Signal peptide of gaussia luciferase
atgggagtgaaagttctttttgcccttatttgtattgctgtggccgaggcc

SEQ ID NO: 14 - DNA, 45 bp, Unknown
  Forward primer for mutagenesis of N1365A of PICP
atgtccaccgaggcctcccaggccatcacctaccactgcaagaac

SEQ ID NO: 15 - DNA, 45 bp, Unknown
  Reverse primer for mutagenesis of N1365A of PICP
gttcttgcagtggtaggtgatggcctgggaggcctcggtggacat